(19) United States
(12) Patent Application Publication
(10) Pub. No.: US 2008/0275027 A1
(43) Pub. Date: Nov. 6, 2008

(54) Piperazinone Derivatives Useful as Histamine H3 Receptor Antagonists and/or Inverse Agonists

(76) Inventors: Rachael Ann Ancliff, Hertfordshire (GB); Mark James Bamford, Essex (GB); Simon Teanby Hodgson, Hertfordshire (GB); Christopher Allan Parr, Essex (GB); Panayiotic Alexandrou Procopiou, Hertfordshire (GB); David Matthew Wilson, Essex (GB); Michael Woodrow, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/995,929

(22) PCT Filed: Jul. 17, 2006
(86) PCT No.: PCT/EP2006/007036

§ 371 Date: Jul. 2, 2008

(30) Foreign Application Priority Data
Jul. 19, 2005 (GB) 0514812.7

(51) Int. Cl. C07D 241/08; A61K 31/497; C07D 401/12; A61P 37/00; A61P 37/08; A61K 31/55; C07D 403/12

(52) U.S. Cl. 514/217.05; 514/253.12; 544/365; 544/383; 514/255.01; 514/254.01; 544/372; 540/598

(57) ABSTRACT

The invention relates to compounds of formula (I) or salts and solvates thereof, processes for their preparation, to compositions containing them and to their use in the treatment of various disorders, such as allergic rhinitis:

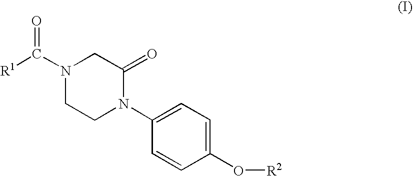

wherein R¹ and R² are as defined in the specification.

The present invention relates to compounds, processes for their preparation, compositions containing them and to their use in the treatment of various disorders, in particular inflammatory and/or allergic disorders of the respiratory tract.

Allergic rhinitis, pulmonary inflammation and congestion are medical conditions that are often associated with other conditions such as asthma, chronic obstructive pulmonary disease (COPD), seasonal allergic rhinitis and perennial allergic rhinitis. In general, these conditions are mediated, at least in part, by inflammation associated with the release of histamine from various cells, in particular mast cells.

Allergic rhinitis, also known as ‘hay fever’ affects a large proportion of the population worldwide. There are two types of allergic rhinitis, seasonal and perennial. The clinical symptoms of seasonal allergic rhinitis, typically include nasal itching and irritation, sneezing and watery rhinorrhea which is often accompanied by nasal congestion. The clinical symptoms of perennial allergic rhinitis are similar except that nasal blockage may be more pronounced. Either type of allergic rhinitis may also cause other symptoms such as itching of the throat and/or eyes, epiphora and oedema around the eyes. The symptoms of allergic rhinitis may vary in intensity from the nuisance level to debilitating.

Allergic rhinitis and other allergic conditions are associated with the release of histamine from various cell types but particularly mast cells. The physiological effects of histamine are classically mediated by three receptor subtypes, termed H1, H2 and H3. H1 receptors are widely distributed throughout the CNS and periphery, and are involved in wakefulness and acute inflammation. H2 receptors mediate gastric acid secretion in response to histamine. H3 receptors are present on the nerve endings in both the CNS and periphery and mediate inhibition of neurotransmitter release [Hill et al., Pharmacol. Rev. 49:253-278 (1997)]. Recently, a fourth member of the histamine receptor family has been identified, termed the H4 receptor [Hough, Mol. Pharmacol., 59:415-419, (2001)]. Whilst the distribution of the H4 receptor appears to be restricted to cells of the immune and inflammatory systems, a physiological role for this receptor remains to be identified.

The activation of H1 receptors in blood vessels and nerve endings are responsible for many of the symptoms of allergic rhinitis, which include itching, sneezing, and the production of watery rhinorrhea. Antihistamine compounds, i.e. drugs which are selective H1 receptor antagonists such as chlorphenyramine and cetirizine, are effective in treating the itching, sneezing and rhinorrhea associated with allergic rhinitis, but are not effective against the nasal congestion symptoms [Aaronson, Ann. Allergy, 67:541-547, (1991)].

Histamine H3 receptors are expressed widely on both CNS and peripheral nerve endings and mediate the inhibition of neurotransmitter release. In vitro electrical stimulation of peripheral sympathetic nerves in isolated human saphenous vein results in an increase in noradrenaline release and smooth muscle contraction, which can be inhibited by histamine H3 receptor agonists [Molderings et al., Naunyn-Schmiedeberg's Arch. Pharmacol., 346:46-50, (1992); Valentine et al., Eur. J. Pharmacol., 366:73-78, (1999)]. H3 receptor agonists also inhibit the effect of sympathetic nerve activation on vascular tone in porcine nasal mucosa [Varty & Hey. Eur. J. Pharmacol., 452:339-345, (2002)]. In vivo, H3 receptor agonists inhibit the decrease in nasal airway resistance produced by sympathetic nerve activation [Hey et al., Arzneim-Forsch Drug Res., 48:881-888 (1998)]. Activation of histamine H3 receptors in human nasal mucosa inhibits sympathetic vasoconstriction [Varty et al., Eur. J. Pharmacol., 484:83-89, (2004)]. Furthermore, H3 receptor antagonists in combination with histamine H1 receptor antagonists have been shown to reverse the effects of mast cell activation on nasal airway resistance and nasal cavity volume, an index of nasal congestion [Mcleod et al., Am. J. Rhinol., 13:391-399, (1999)], and further evidence for the contribution of H3 receptors to histamine-induced nasal blockage is provided by histamine nasal challenge studies performed on normal human subjects [Taylor-Clark et al., Br. J. Pharmacol., 1-8 (2005)].

The present invention relates to compounds (or salts thereof) that are histamine H3 antagonists and/or inverse agonists. Such compounds (or salts thereof) may be useful in the treatment of various disorders in particular inflammatory and/or allergic disorders, such as inflammatory and/or allergic disorders of the respiratory tract, for example allergic rhinitis, that are associated with the release of histamine from cells such as mast cells. Compounds of the invention (or salts thereof) may show an improved profile over known H3 antagonists/inverse agonists in that they may possess one or more of the following properties:

(i) potent H3 antagonist/inverse agonist activity;
(ii) at least 100 fold more selective for the H3 receptor over the H1 receptor;
(iii) low CNS penetration;
(iv) improved bioavailability; and
(v) lower clearance and/or longer half-life in blood.

Compounds having such a profile may be orally effective, and/or capable of once daily administration and/or further may have an improved side effect profile compared with other existing therapies.

Thus the present invention provides, in a first aspect, a compound of formula (I) or a salt thereof:

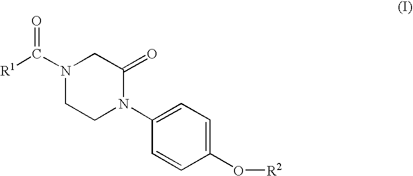

in which
R¹ represents —C₁₋₆alkyl, —C₁₋₆alkoxy, —C₃₋₈cycloalkyl (optionally substituted by C₁₋₆alkyl), heterocyclyl, aryl, heteroaryl, and -aryl-heteroaryl,
wherein independently each of said heterocyclyl, aryl, heteroaryl, and -aryl-heteroaryl of R¹ may be optionally substituted by one or two substituents which may be the same or different selected from C₁₋₆alkyl, (optionally substituted by COOR³ wherein R³ is C₁₋₆alkyl or hydrogen), C₁₋₆alkoxy (optionally substituted by COOR⁴ wherein R⁴ is C₁₋₆alkyl or hydrogen), cyano, oxo, halogen, C₁₋₆alkylsulfonyl, —C₁₋₄alkylCONR⁵R⁶ wherein R⁵ and R⁶ independently represent hydrogen, C₁₋₆alkyl, C₁₋₆alkoxy, C₁₋₆alkylC₁₋₆ alkoxy, or together NR⁵R⁶ may form a 4- to 7-membered non-aromatic heterocyclic ring (optionally containing an O or S atom and optionally substituted by C₁₋₆alkyl, halogen or C₁₋₆alkoxy), —CONR⁷R⁸ wherein R⁷ and R³ independently represent hydrogen, C₁₋₆alkyl, C₁₋₆alkoxy, C₁₋₆alkylC₁₋₆alkoxy, or together NR⁷R⁸ may form a 4- to 7-membered non-aromatic heterocyclic ring (optionally containing an 0 or an S atom and optionally substituted by C₁₋₆alkyl, halogen or C₁₋₆alkoxy);
R² represents —(CH₂)ₓ—NR⁹R¹⁰ in which NR⁹R¹⁰ represents an N-linked nitrogen containing heterocyclyl ring (optionally substituted by one or two substituents selected from trifluoromethyl or C₁₋₆alkyl), and x is 2, 3 or 4, or
R² represents the group:

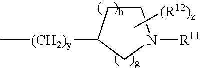

wherein R¹¹ represents C₁₋₆alkyl, C₃₋₈cycloalkyl or C₁₋₆alkyl C₃₋₈cycloalkyl, R¹² represents trifluoromethyl or C₁₋₆alkyl, z is 0 or 1, y is 0 or 1, and g is 0, 1, or 2 and h is 0, 1, 2, or 3 such that g and h cannot both be 0;
with the proviso that the compound is not 4-[(2,4-difluorophenyl)carbonyl]-1-[4-({3-[2-methyl-1-pyrrolidinyl]propyl}oxy)phenyl]-2-piperazinone, or a salt thereof.

In another embodiment of the present invention R¹ may be optionally substituted by one or two substituents which may be the same or different selected from C₁₋₆alkyl, (optionally substituted by COOR³ wherein R³ is C₁₋₆alkyl or hydrogen), C₁₋₆alkoxy (optionally substituted by COOR⁴ wherein R⁴ is C₁₋₆alkyl or hydrogen), cyano, oxo, C₁₋₆alkylsulfonyl, —C₁₋₆alkylCONR⁵R⁶ wherein R⁵ and R⁶ independently represent hydrogen, C₁₋₆alkyl, C₁₋₆alkoxy, C₁₋₆alkylC₁₋₆alkoxy, or together NR⁵R⁶ may form a 4- to 7-membered non-aromatic heterocyclic ring (optionally containing an O or S atom and optionally substituted by C₁₋₆alkyl, halogen or C₁₋₆alkoxy), —CONR⁷R⁸ wherein R⁷ and R⁸ independently represent hydrogen, C₁₋₆alkyl, C₁₋₆alkoxy, C₁₋₆ alkylC₁₋₆alkoxy, or together NR⁷R⁸ may form a 4- to 7-membered non-aromatic heterocyclic ring (optionally containing an 0 or an S atom and optionally substituted by C₁₋₆alkyl, halogen or C₁₋₆alkoxy).

C₁₋₆alkyl, whether alone or as part of another group, may be straight chain or branched and C₁₋₆alkoxy shall be interpreted similarly. Representative examples include methyl, ethyl, n-propyl, iso-propyl, n-butyl, sec-butyl, iso-butyl, t-butyl, n-pentyl, neo-pentyl and n-hexyl. Particular alkyl and alkoxy groups are C₁₋₃ alkyl and C₁₋₃ alkoxy.

C₃₋₈cycloalkyl refers to a non-aromatic cyclic hydrocarbon ring having from three to eight carbon atoms. Representative examples include, but are not limited to, cyclopropyl, cyclobutyl, cyclopentyl, cyclohexyl and cycloheptyl. Exemplary C₃₋₈ cycloalkyl groups are cyclopropyl, cyclobutyl, cyclopentyl and cyclohexyl.

The term “halogen” is used herein to describe, unless otherwise stated, a group selected from fluorine, chlorine, bromine or iodine. Particular halogen substituents include fluorine and chlorine.

The term “aryl” includes single and fused aromatic rings. Representative aryl groups include phenyl and naphthyl. Naphthyl is intended to denote both naphth-1-yl and naphth-2-yl.

The term “heterocyclyl” is intended to mean a 4-7 membered monocyclic saturated or partially unsaturated ring or a 4-7 membered saturated or partially unsaturated ring fused to a benzene ring containing 1 to 3 heteroatoms selected from O, N, SO₂ and S. Suitable examples of such monocyclic rings include azetidinyl, pyrrolidinyl, piperidinyl, piperazinyl, morpholinyl, thiomorpholinyl, tetrahydrofuranyl, diazepanyl, azepanyl and azocanyl. Suitable examples of benzofused heterocylic rings include indolinyl, isoindolinyl, benzodioxolyl, dihydroquinolinyl, dihydroisoquinolinyl, dihydrobenzothiopyranyl and dihydrobenzothiopyranyl-1-dioxide.

Particular heterocyclyl groups include azetidinyl, pyrrolidinyl, morpholinyl, piperidinyl, dihydroquinolinyl, dihydroisoquinolinyl, dihydrobenzothiopyranyl dihydrobenzothiopyranyl-1-dioxide and benzodioxolyl. Exemplary heterocyclyl groups are pyrrolidinyl, dihydrobenzothiopyranyl-1-dioxide and benzodioxolyl.

The term “heteroaryl” is intended to mean a 5-7 membered monocyclic aromatic or a 8-11 membered bicyclic aromatic ring containing 1 to 3 heteroatoms selected from oxygen, nitrogen and sulphur. Suitable examples of such monocyclic aromatic rings include thienyl, furyl, pyrrolyl, triazolyl, imidazolyl, oxazolyl, thiazolyl, oxadiazolyl, and isothiazolyl, isoxazolyl, thiadiazolyl, pyrazolyl, pyrimidyl, pyridazinyl, pyrazinyl, pyridyl, and triazolyl. Suitable examples of such 8-11 membered bicyclic aromatic rings include furopyridinyl and pyrazolopyrimidyl, and benzofused aromatic rings such as quinolinyl, isoquinolinyl, quinazolinyl, quinoxalinyl, cinnolinyl, naphthyridinyl, indolyl, indazolyl, pyrrolopyridinyl, benzofuranyl, benzothienyl, benzimidazolyl, benzoxazolyl, benzisoxazolyl, benzothiazolyl, benzisothiazolyl, benzotriazolyl, benzoxadiazolyl, benzothiadiazolyl and the like.

Particular heteroaryl groups include pyridyl, pyrazolyl, isoxazolyl, triazolyl, pyrazolopyrimidyl, indolyl, quinolinyl, benzothiazolyl and benzotriazolyl.

Representative examples of —(CH₂)ₓ—NR⁹R¹⁰ include those in which x is 2, 3 or 4 (e.g. 3), and NR⁹R¹⁰ is pyrrolidinyl, piperidinyl or azepinyl optionally substituted by one or two (e.g. none or one) substituents(s) selected from trifluoromethyl and C₁₋₆alkyl (such as C₁₋₃alkyl e.g. methyl).

Representative examples of:

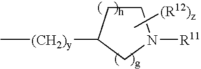

include those in which y is 0 or 1 (such as 0), h is 1 or 2 and g is 1 or 2 (e.g. pyrrolidinyl or piperidinyl), R¹¹ is C₁₋₆alkyl (such as C₁₋₃alkyl e.g. isopropyl), C₃₋₈cycloalkyl (e.g. cyclobutyl and cyclopentyl) or C₁₋₆alkylC₃₋₈cycloalkyl (e.g. C₁₋₃alkylC₅₋₆cycloalkyl), R¹² represents trifluoromethyl or C₁₋₆alkyl, and z is 0 or 1 (e.g. z is 0).

In another embodiment of the invention R¹ represents —C₃₋₆alkyl, —C₃₋₆cycloalkyl (optionally substituted by methyl or ethyl, e.g. methyl), phenyl, naphthyl, pyridyl, pyrazolyl, oxazolyl, isoxazolyl, triazolyl, pyrazolopyrimidyl, indolyl, quinolinyl, benzothiazolyl, benzotriazolyl, dihydrobenzothiopyranyl-1-dioxide, pyrrolidinyl, benzodioxolyl or -phenyl-oxazolyl.

In a further embodiment of the invention R¹ may be unsubstituted or may be optionally substituted with 1 or 2 substituent independently selected from C₁₋₃alkyl (optionally substituted by COOR³ wherein R³ is hydrogen or C₁₋₃alkyl) e.g. methyl, ethyl or iso-propyl; C₁₋₃alkoxy (optionally substituted by COOR⁴ wherein R⁴ is hydrogen or C₁₋₃alkyl) e.g. methoxy or —OCH₂C(O)OCH₃; cyano; oxo; halogen e.g. chlorine or fluorine; C₁₋₃alkylsulfonyl e.g. methylsulfonyl; —C₁₋₃alkylCONR⁵R⁶ wherein R⁵ and R⁶ independently represent hydrogen or C₁₋₃alkyl or together NR⁵R⁶ may form azetidinyl, pyrrolidinyl or morpholinyl e.g. —CH₂C(O)NH₂; and —CONR⁷R⁸ wherein R⁷ and R⁸ independently represent hydrogen, C₁₋₃alkyl e.g. methyl or C₁₋₃alkylC₁₋₃alkoxy e.g. —CH₂CH₂OCH₃ or together NR⁷R⁸ may form azetidinyl, pyrrolidinyl or morpholinyl.

It is to be understood that the present invention covers all combinations of particular, embodiments, exemplary, representative groups and substituents etc. described hereinabove.

Particular compounds according to the invention include Examples E1 to E180 and salts thereof, particularly pharmaceutically acceptable salts or solvates.

It is to be further understood that references hereinafter to a compound of formula (I) or the compounds of the invention means a compound of formula (I) as the free base, or as a salt, or as a solvate.

The compounds of the present invention may be in the form of and/or may be administered as a pharmaceutically acceptable salt. A pharmaceutically acceptable salt may be readily prepared by using a desired acid as appropriate. The salt may precipitate from solution and be collected by filtration or may be recovered by evaporation of the solvent. Pharmaceutically acceptable salts include acid addition salts and base addition salts. For a review on suitable salts see Berge et al., J. Pharm. Sci., 66:1-19 (1977). Typically, a pharmaceutically acceptable acid addition salt can be formed by reaction of a compound of formula (I) with a suitable inorganic or organic acid (such as hydrobromic, hydrochloric, formic, sulfuric, nitric, phosphoric, succinic, maleic, acetic, fumaric, citric, tartaric, benzoic, p-toluenesulfonic, methanesulfonic or naphthalenesulfonic acid), optionally in a suitable solvent such as an organic solvent, to give the salt which is usually isolated for example by crystallisation and filtration. Thus, a pharmaceutically acceptable acid addition salt of a compound of formula (I) can be for example a hydrobromide, hydrochloride, formate, sulfate, nitrate, phosphate, succinate, maleate, acetate, fumarate, citrate, tartrate, benzoate, p-toluenesulfonate, methanesulfonate or naphthalenesulfonate salt.

Suitable pharmaceutically acceptable base salts include ammonium salts, alkali metal salts such as those of sodium and potassium, alkaline earth metal salts such as those of calcium and magnesium, and salts with organic bases, including salts of primary, secondary and tertiary amines such as isopropylamine, diethylamine, ethanolamine, trimethylamine, dicyclohexylamine and N-methyl-D-glucamine.

Other non-pharmaceutically acceptable salts, e.g. oxalates or trifluoroacetates, may be used, for example in the isolation of compounds of the invention, and are included within the scope of this invention. The invention includes within its scope all possible stoichiometric and non-stoichiometric forms of the salts of the compounds of formula (I).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or solvents with a high propensity to form hydrogen bonds such as water, xylene, N-methylpyrrolidinone methanol may be used to form solvates. Methods for identification of solvates include, but are not limited to, NMR and microanalysis. Solvates of the compound of the invention are within the scope of the invention.

It will be appreciated that compounds of formula (I) may possess one or more asymmetric carbon atoms so that optical isomers e.g. enantiomers or diastereoisomers may be formed. The present invention encompasses all optical isomers of the compounds of formula (I) whether as individual isomers isolated such as to be substantially free of the other isomer (i.e. pure) or as mixtures thereof (i.e. racemates and racemic mixtures). An individual isomer isolated such as to be substantially free of the other isomer (i.e. pure) may be isolated such that less than about 10%, particularly less than about 1%, for example less than about 0.1% of the other isomer is present.

Certain compounds of formula (I) may exist in one of several tautomeric forms. It will be understood that the present invention encompasses all tautomers of the compounds of formula (I) whether as individual tautomers or as mixtures thereof.

The compounds of formula (I) may be in crystalline or amorphous form. Furthermore, some of the crystalline forms of the compounds of formula (I) may exist as polymorphs, which are included within the scope of the present invention. The most thermodynamically stable polymorphic form of compounds of formula (I) are of particular interest.

Polymorphic forms of compounds of formula (I) may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (NMR).

It is to be further understood that references hereinafter to compounds of the invention or to compounds of formula (I) means a compound of formula (I) as the free base, or as a salt.

It will be appreciated from the foregoing that included within the scope of the invention are all solvates, hydrates, complexes, isomers and polymorphic forms of the compound of the invention and salts thereof.

The present invention also provides processes for the preparation of a compound of formula (I) or a salt thereof.

Thus, in a first process (A) a compound of formula (I) or a salt thereof may be prepared by reacting a compound of formula (II):

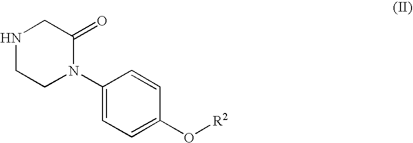

in which R² is as defined above for a compound of formula (I) or a salt thereof,
with a compound of formula (III)

R¹COOH

in which R¹ is as defined above for a compound of formula (I).

The reaction may be carried out in the presence of a suitable base such as triethylamine or polymer supported diisopropylethylamine (PS-DIPEA) and a suitable coupling agent such as O-(benzotriazol-1-yl)-N,N,N′-tetramethyluronium tetrafluoroborate (TBTU) or 2-(1H-benzotriazol-1-yl)-1,1,3,3-tetramethyluronium hexafluorophosphate (HBTU) in an appropriate solvent such as dichloromethane or N,N-dimethylformamide.

Compounds of formula (II) wherein R² represents —(CH₂)ₓ—NR⁹R¹⁰ may be prepared by removal of the protecting group from a compound of formula (III):

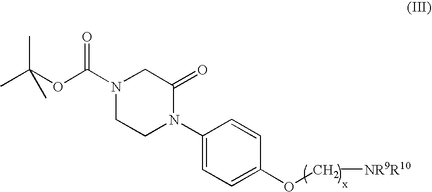

in which x and NR⁹R¹⁰ are as defined above for a compound of formula (I).

Deprotection may be achieved by hydrolysis using an acid such as trifluoroacetic acid in dichloromethane or 4M hydrogen chloride in dioxane.

Compounds of formula (III) may be prepared by reacting a compound of formula (IV):

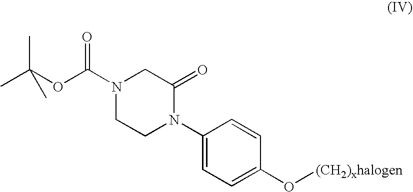

wherein x is as defined for a compound of formula (I) and halogen is as defined herein, with an appropriate amine. The reaction may be carried out in the presence of a suitable catalyst such as potassium iodide or sodium iodide, optionally in the presence of a suitable base such as potassium carbonate, in a suitable polar solvent such as 2-butanone and at an elevated temperature e.g. 60° C.-90° C.

A compound of formula (IV) may be prepared by reacting a compound of formula (V):

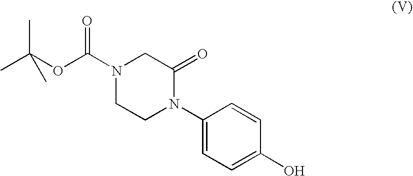

with an appropriate dihalogenoalkane such as halogen(CH₂)ₓhalogen wherein halogen and x are as defined above, in the presence of a suitable base such as potassium carbonate and a suitable polar solvent such as 2-butanone. Suitable examples of halogen(CH₂)ₓhalogen include 1,3-dibromopropane and 1-bromo-3-chloropropane (commercially available, for example, from Aldrich).

A compound of formula (V) may be prepared in accordance with the following general reaction scheme 1:

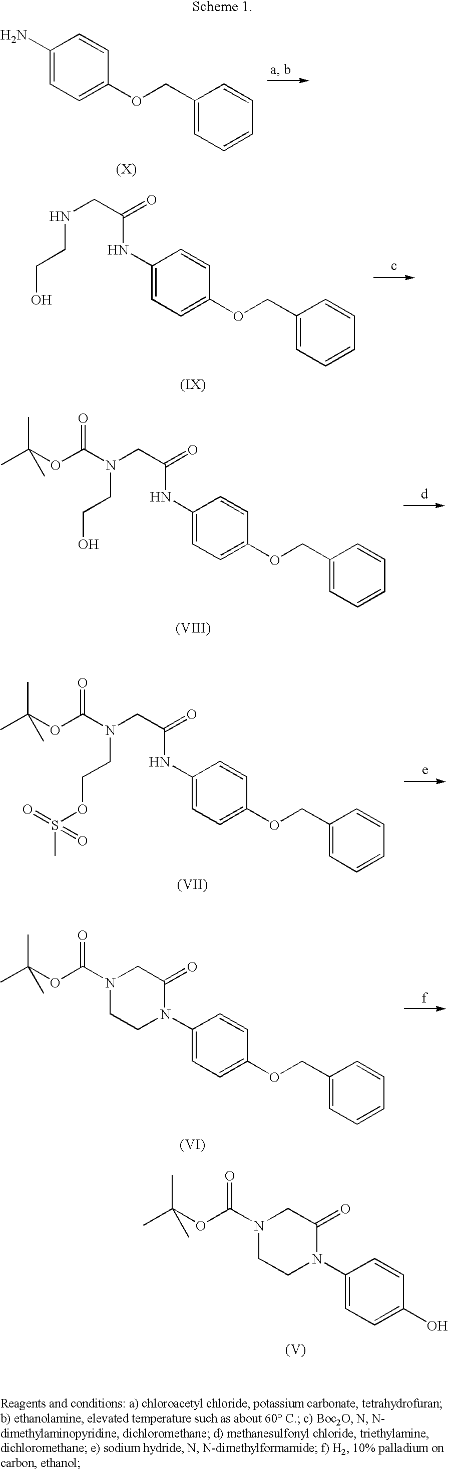

Compounds of formula (II) in which R² represents the group

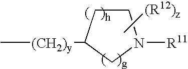

as defined in formula (I) wherein y, z, h, g, R¹¹ and R¹² are as described above may be prepared from a compound (XI) by cleavage of the amine protecting group e.g. by hydrogenolysis over palladium on carbon in ethanol.

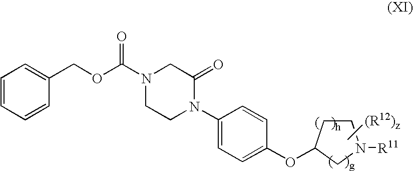

Compound (XI) may be prepared from compound (XII):

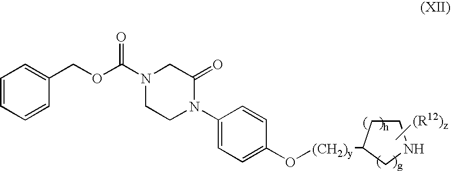

by reductive amination with an appropriate ketone e.g. cyclobutanone, cyclopentanone or acetone to introduce group R¹¹ and in the presence of a reducing agent e.g. sodium triacetoxyborohydride in dichloromethane. Alternatively, introduction of group R¹¹ may be effected using the appropriate alkyl halide such as alkyl bromide or alkyl iodide e.g. cyclobutyl bromide, cyclopentyl bromide or iso-propyl bromide or using sulfonate ester e.g. methanesulfonate or tosylate ester, optionally in the presence of a catalyst such as potassium iodide in a suitable solvent such as 2-butanone or DMF.

Compound (XII) may be prepared in accordance with the general reaction scheme 2:

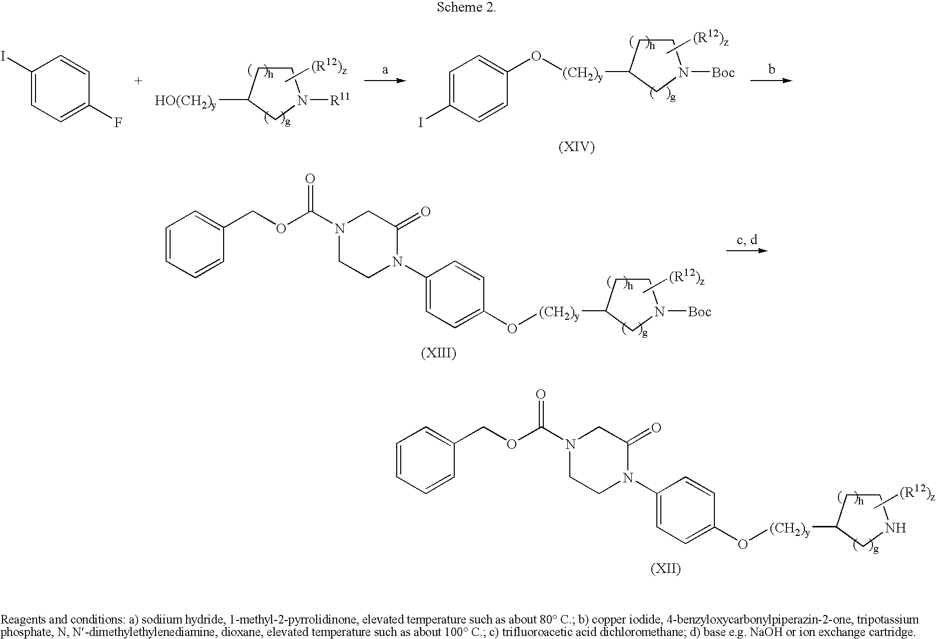

It will be appreciated that other suitable reagents may be substituted for the solvents, bases, protecting groups etc. specifically mentioned herein. Further it will be appreciated that reaction conditions such as temperature, reaction times etc. may be varied from the specific conditions described herein, as appropriate, depending on the specific reagents chosen.

Thus, compound (IX), for example, may be protected with benzyloxycarbonyl or 2′,2′,2′-trichloroethoxycarbonyl groups. The activation of compound (VIII) may be achieved by reaction with tosyl chloride or triflic anhydride in pyridine or in dichloromethane and the presence of a base such as diisopropylethylamine to provide the corresponding p-toluenesulfonate ester or the trifluoromethanesulfonate ester. Cyclisation of compound (VII) to compound (VI) may be achieved in the presence of other kinetic bases such as sodium bis(trimethylsilyl)amide in tetrahydrofurane. The cleavage of the protecting group from (III) may provide a salt e.g. trifluoroacetic acid salt in the case of clevage of the BOC protecting group with TFA. The free base can then be isolated following ion exchange chromatography on SCX-2 cartridge. Alternatively the salt can be used in the acylation reaction in the presence of a base such as triethylamine, diisopropylethylamine or polymer-supported diisopropylethylamine.

Alternatively compound (XIV) may be prepared from the same alcohol and 4-iodophenol under Mitsunobu conditions i.e. in the presence of a phosphine such as triphenylphosphine and a diazodicarboxylate such as di-tert-butyl azodicarboxylate.

Other protecting groups that may be employed in the synthetic routes described herein and the means for their removal can be found in T. W. Greene ‘Protective Groups in Organic Synthesis’ (3rd edition, J. Wiley and Sons, 1999). Suitable amine protecting groups include sulphonyl (e.g. tosyl), acyl (e.g. acetyl, 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrogen chloride in dioxan or trifluoroacetic acid in dichloromethane) or reductively (e.g. hydrogenolysis of a benzyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis or a solid phase resin bound benzyl group, such as a Merrifield resin bound 2,6-dimethoxybenzyl group (Ellman linker), which may be removed by acid catalysed hydrolysis, for example with trifluoroacetic acid.

In a second process (B) a compound of formula (I) or a salt thereof may be prepared by interconversion from other compounds of formula (I) using conventional interconversion procedures which include but are not limited to hydrogenation, epimerisation, oxidation, reduction, alkylation, nucleophilic or electrophilic aromatic substitution.

According to a third process (C), a salt of a compound of formula (I) may be prepared by exchange of counterions, or precipitation of the desired salt from the free base.

Further it will be appreciated that the R and S enantiomers may be isolated from the racemate by conventional resolution methods such as preparative HPLC involving a chiral stationary phase, by resolution using fractional crystallisation of a salt of the free base with a chiral acid, by chemical conversion to a diastereoisomer using a chiral auxiliary followed by chromatographic separation of the isomers and then removal of the chiral auxiliary and regeneration of the pure enantiomer, or by total asymmetric synthesis.

It will be appreciated that all novel intermediates used to prepare compounds of the invention form yet another aspect of the present invention.

Examples of disease states in which compounds of formula (I), or pharmaceutically acceptable salts thereof may have potentially beneficial anti-inflammatory and/or anti-allergic effects include diseases of the respiratory tract such as bronchitis (including chronic bronchitis), asthma (including allergen-induced asthmatic reactions), chronic obstructive pulmonary disease (COPD), cystic fibrosis, sinusitis and allergic rhinitis (seasonal and perennial). Other disease states include diseases of the gastrointestinal tract such as intestinal inflammatory diseases including inflammatory bowel disease (e.g. Crohn's disease or ulcerative colitis) and intestinal inflammatory diseases secondary to radiation exposure or allergen exposure.

Furthermore, compounds of the invention may be used to treat nephritis, skin diseases such as psoriasis, eczema, allergic dermatitis and hypersensitivity reactions.

The compounds of the invention may also be of use in the treatment of nasal polyposis, conjunctivitis or pruritis.

Further diseases include inflammatory diseases of the gastrointestinal tract such as inflammatory bowel disease.

A disease of particular interest is allergic rhinitis.

Compounds that are antagonists and/or inverse agonists of the H3 receptor may also be of use in other diseases in which activation of the H3 receptor may be implicated. Such diseases may include non-allergic rhinitis.

It will be appreciated by those skilled in the art that references herein to treatment or therapy extend to prophylaxis as well as the treatment of established conditions.

As mentioned above, compounds of formula (I) are useful as therapeutic agents. There is thus provided, as a further aspect of the invention, a compound of formula (I) or a pharmaceutically acceptable salt thereof for use in therapy.

According to another aspect of the invention, there is provided the use of a compound of formula (I) or a pharmaceutically acceptable salt thereof for the manufacture of a medicament for the treatment of any of the above diseases.

In a further aspect, there is provided a method for the treatment of any of the above diseases, in a human or animal subject in need thereof, which method comprises administering an effective amount of a compound of formula (I) or a pharmaceutically acceptable salt thereof.

When used in therapy, the compounds of formula (I) are usually formulated in a suitable pharmaceutical composition. Such compositions can be prepared using standard procedures.

Thus, the present invention further provides a pharmaceutical composition which comprises a compound of formula (I) or a pharmaceutically acceptable salt thereof optionally with one or more pharmaceutically acceptable carriers and/or excipients.

A composition of the invention, which may be prepared by admixture, suitably at ambient temperature and atmospheric pressure, is usually adapted for oral, parenteral or rectal administration and, as such, may be in the form of tablets, capsules, oral liquid preparations, powders, granules, lozenges, reconstitutable powders, injectable or infusible solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and acceptable wetting agents. The tablets may be coated according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colorants.

For parenteral administration, fluid unit dosage forms are prepared utilising a compound of the invention or pharmaceutically acceptable salt thereof and a sterile vehicle. The compound, depending on the vehicle and concentration used, can be either suspended or dissolved in the vehicle. In preparing solutions, the compound can be dissolved for injection and filter sterilised before filling into a suitable vial or ampoule and sealing. Advantageously, adjuvants such as a local anaesthetic, preservatives and buffering agents are dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilisation cannot be accomplished by filtration. The compound can be sterilised by exposure to ethylene oxide before suspension in a sterile vehicle. A surfactant or wetting agent may be included in the composition to facilitate uniform distribution of the compound.

The composition may contain from about 0.1% to 99% by weight, such as from about 10 to 60% by weight, of the active material, depending on the method of administration. The dose of the compound used in the treatment of the aforementioned disorders will vary in the usual way with the seriousness of the disorders, the weight of the sufferer, and other similar factors. However, as a general guide suitable unit doses may be about 0.05 to 1000 mg, more suitably about 1.0 to 200 mg, and such unit doses may be administered more than once a day, for example two or three a day. Such therapy may extend for a number of weeks or months. In one embodiment, compounds and compositions according to the invention are suitable for oral administration and/or are capable of once daily administration.

The compounds and compositions according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents (e.g. antibiotics, antivirals), or antihistamines. The invention thus provides, in a further aspect, a combination comprising a compound of formula (I) or a pharmaceutically acceptable salt thereof, together with one or more other therapeutically active agents, for example selected from an anti-inflammatory agent (for example another corticosteroid or an NSAID), an anticholinergic agent, a β₂-adrenoreceptor agonist, an antiinfective agent (e.g. an antibiotic or an antiviral), or an antihistamine. Combinations comprising a compound of formula (I) or a pharmaceutically acceptable salt thereof, together with a β₂-adrenoreceptor agonist, and/or an anticholinergic, and/or a PDE-4 inhibitor form yet another aspect of the invention. The combinations of the invention may comprise one or two other therapeutic agents, and may optionally include one or more pharmaceutically acceptable carriers and/or excipients as desired.

It will be clear to a person skilled in the art that, where appropriate, the other therapeutic ingredient(s) may be used in the form of salts, (e.g. as alkali metal or amine salts or as acid addition salts), or prodrugs, or as esters (e.g. lower alkyl esters), or as solvates (e.g. hydrates) to optimise the activity and/or stability and/or physical characteristics (e.g. solubility) of the therapeutic ingredient. It will be clear also that where appropriate, the therapeutic ingredients may be used in optically pure form.

Examples of β₂-adrenoreceptor agonists include salmeterol (e.g. as racemate or a single enantiomer such as the R-enantiomer or the S-enantiomer), salbutamol (e.g. as racemate or a single enantiomer such as the R-enantiomer), formoterol (e.g. as racemate or a single enantiomer such as the R-enantiomer), salmefamol, fenoterol carmoterol, etanterol, naminterol, clenbuterol, pirbuterol, flerbuterol, reproterol, bambuterol, indacaterol, terbutaline and salts thereof, for example the xinafoate (1-hydroxy-2-naphthalenecarboxylate) salt of salmeterol, the sulphate salt or free base of salbutamol or the fumarate salt of formoterol. Combinations comprising a compound of the invention together with a longer-acting β2-adrenoreceptor agonist, which provides effective bronchodilation for about 12 hours or longer, may be of particular interest.

Other β₂-adrenoreceptor agonists include those described in WO 02/066422, WO 02/070490, WO 02/076933, WO 03/024439, WO 03/072539, WO 03/091204, WO 04/016578, WO 2004/022547, WO 2004/037807, WO 2004/037773, WO 2004/037768, WO 2004/039762, WO 2004/039766, WO01/42193 and WO03/042160.

Exemplary β₂-adrenoreceptor agonists include:
3-(4-{[6-({(2R)-2-hydroxy-2-[4-hydroxy-3-(hydroxymethyl)phenyl]ethyl}amino)hexyl]oxy}butyl)benzenesulfonamide;
3-(3-{[7-({(2R)-2-hydroxy-2-[4-hydroxy-3-hydroxymethyl)phenyl]ethyl}-amino)heptyl]oxy}propyl)benzenesulfonamide;
4-{(1R)-2-[(6-{2-[(2,6-dichlorobenzyl) oxy]ethoxy}hexyl)amino]-1-hydroxyethyl}-2-(hydroxymethyl)phenol;
4-{(1R)-2-[(6-{4-[3-(cyclopentylsulfonyl)phenyl]butoxy}hexyl)amino]-1-hydroxyethyl}-2-(hydroxymethyl)phenol;
N-[2-hydroxyl-5-[(1R)-1-hydroxy-2-[[2-4-[[(2R)-2-hydroxy-2-phenylethyl]amino]phenyl]ethyl]amino]ethyl]phenyl]formamide;
N-2{2-[4-(3-phenyl-4-methoxyphenyl)aminophenyl]ethyl}-2-hydroxy-2-(8-hydroxy-2(1H)-quinolinon-5-yl)ethylamine; and
5-[(R)-2-(2-{4-[4-(2-amino-2-methyl-propoxy)-phenylamino]-phenyl}-ethylamino)-1-hydroxy-ethyl]-8-hydroxy-1H-quinolin-2-one.

Anti-inflammatory agents include corticosteroids. Corticosteroids which may be used in combination with the compound of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester, 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (e.g. the 17-propionate ester or the 17,21-dipropionate ester), budesonide, flunisolide, mometasone esters (e.g. the furoate ester), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. Corticosteroids that may be of interest include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester and 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17β-(2,2,3,3-tetramethycyclopropylcarbonyl)oxy-androsta-1,4-diene-17α-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, especially 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Non-steroidal compounds having glucocorticoid agonism that may possess selectivity for transrepression over transactivation and that may be useful in combination therapy include those covered in the following patents: WO03/082827, WO01/10143, WO98/54159, WO04/005229, WO04/009016, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277.

Anti-inflammatory agents include non-steroidal anti-inflammatory drugs (NSAID's). NSAID's include sodium cromoglycate, nedocromil sodium, phosphodiesterase (PDE) inhibitors (e.g. theophylline, PDE4 inhibitors or mixed PDE3/PDE4 inhibitors), leukotriene antagonists, inhibitors of leukotriene synthesis (e.g. montelukast), iNOS inhibitors, tryptase and elastase inhibitors, beta-2 integrin antagonists and adenosine receptor agonists or antagonists (e.g. adenosine 2a agonists), cytokine antagonists (e.g. chemokine antagonists, such as a CCR3 antagonist) or inhibitors of cytokine synthesis, or 5-lipoxygenase inhibitors. iNOS inhibitors include those disclosed in WO93/13055, WO98/30537, WO02/50021, WO95/34534 and WO99/62875. CCR3 inhibitors include those disclosed in WO02/26722. Adenosine 2a agonists include those disclosed in WO2005/116037.

The PDE4-specific inhibitor useful in combinations of the invention may include any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

PDE4 inhibitors include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Also, cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996.

Other PDE4 inhibitors include AWD-12-281 from Elbion (Hofgen, N. et al., 15th EFMC Int Symp. Med. Chem. (September 6-10, Edinburgh) 1998, Abst. P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al., Eur. Resp. J. [Ann. Cong. Eur. Resp. Soc. (September 19-23, Geneva) 1998] 1998, 12 (Suppl. 28): Abst. P2393); roflumilast (CAS reference No 162401-32-3) and a pthalazinone (WO99/47505) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al., J. Pharmacol. Exp. Ther., 284(1):162, (1998)), and T2585.

Further compounds of interest are disclosed in the published international patent application WO04/024728 (Glaxo Group Ltd), PCT/EP2003/014867 (Glaxo Group Ltd) and PCT/EP2004/005494 (Glaxo Group Ltd).

Anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (e.g. as the bromide, CAS 22254-24-6, sold under the name Atrovent), oxitropium (e.g. as the bromide, CAS 30286-75-0) and tiotropium (e.g. as the bromide, CAS 136310-93-5, sold under the name Spiriva). Also of interest are revatropate (e.g. as the hydrobromide, CAS 262586-79-8) and LAS-34273 which is disclosed in WO01/04118. Exemplary compounds for oral administration include pirenzepine (CAS 28797-61-7), darifenacin (CAS 133099-04-4, or CAS 133099-07-7 for the hydrobromide sold under the name Enablex), oxybutynin (CAS 5633-20-5, sold under the name Ditropan), terodiline (CAS 15793-40-5), tolterodine (CAS 124937-51-5, or CAS 124937-52-6 for the tartrate, sold under the name Detrol), otilonium (e.g. as the bromide, CAS 26095-59-0, sold under the name Spasmomen), trospium chloride (CAS 10405-02-4) and solifenacin (CAS 242478-37-1, or CAS 242478-38-2 for the succinate also known as YM-905 and sold under the name Vesicare).

Other anticholinergic agents include compounds of formula (XXI), which are disclosed in U.S. patent application 60/487,981:

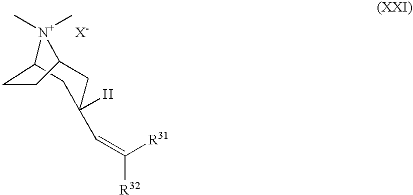

in which the orientation of the alkyl chain attached to the tropane ring may be endo;
R³¹ and R³² are, independently, selected from the group consisting of straight or branched chain lower alkyl groups having, for example, from 1 to 6 carbon atoms, cycloalkyl groups having from 5 to 6 carbon atoms, cycloalkyl-alkyl having 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;
X⁻ represents an anion associated with the positive charge of the N atom. X⁻ may be but is not limited to chloride, bromide, iodide, sulfate, benzene sulfonate, and toluene sulfonate,
including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds of formula (XXII) or (XXIII), which are disclosed in U.S. patent application 60/511,009:

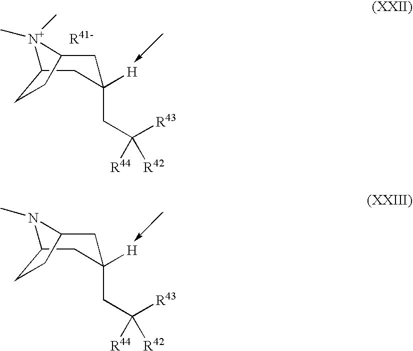

wherein:
the H atom indicated is in the exo position;
R⁴¹ represents an anion associated with the positive charge of the N atom. R⁴¹ may be but is not limited to chloride, bromide, iodide, sulfate, benzene sulfonate and toluene sulfonate;
R⁴² and R⁴³ are independently selected from the group consisting of straight or branched chain lower alkyl groups (having, for example, from 1 to 6 carbon atoms), cycloalkyl groups (having from 5 to 6 carbon atoms), cycloalkyl-alkyl (having 6 to 10 carbon atoms), heterocycloalkyl (having 5 to 6 carbon atoms) and N or O as the heteroatom, heterocycloalkyl-alkyl (having 6 to 10 carbon atoms) and N or O as the heteroatom, aryl, optionally substituted aryl, heteroaryl, and optionally substituted heteroaryl;
R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), —CH₂N(R⁴⁸)CONH(R⁴⁷);
R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;
R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;
R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl, including, for example:
(Endo)-3-(2-methoxy-2,2-di-thiophen-2-yl-ethyl)-8,8-dimethyl-8-azonia-bicyclo[3.2.1]octane iodide;
3-((Endo)-8-methyl-8-aza-bicyclo[3.2.1]oct-3-yl)-2,2-diphenyl-propionitrile;
(Endo)-8-methyl-3-(2,2,2-triphenyl-ethyl)-8-aza-bicyclo[3.2.1]octane;
3-((Endo)-8-methyl-8-aza-bicyclo[3.2.1]oct-3-yl)-2,2-diphenyl-propionamide;
3-((Endo)-8-methyl-8-aza-bicyclo[3.2.1]oct-3-yl)-2,2-diphenyl-propionic acid;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
3-((Endo)-8-methyl-8-aza-bicyclo[3.2.1]oct-3-yl)-2,2-diphenyl-propan-1-ol;
N-Benzyl-3-((endo)-8-methyl-8-aza-bicyclo[3.2.1]oct-3-yl)-2,2-diphenyl-propionamide;
(Endo)-3-(2-carbamoyl-2,2-diphenyl-ethyl)-8,8-dimethyl-8-azonia-bicyclo[3.2.1]octane iodide;
1-Benzyl-3-[3-((endo)-8-methyl-8-aza-bicyclo[3.2.1]oct-3-yl)-2,2-diphenyl-propyl]-urea;
1-Ethyl-3-[3-((endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-acetamide;
N-[3-((Endo)-8-methyl-8-aza-bicyclo[3.2.1]oct-3-yl)-2,2-diphenyl-propyl]-benzamide;
3-((Endo)-8-methyl-8-aza-bicyclo[3.2.1]oct-3-yl)-2,2-di-thiophen-2-yl-propionitrile;
(Endo)-3-(2-cyano-2,2-di-thiophen-2-yl-ethyl)-8,8-dimethyl-8-azonia-bicyclo[3.2.1]octane iodide;
N-[3-((Endo)-8-methyl-8-aza-bicyclo[3.2.1]oct-3-yl)-2,2-diphenyl-propyl]-benzenesulfonamide;
[3-((Endo)-8-methyl-8-aza-bicyclo[3.2.1]oct-3-yl)-2,2-diphenyl-propyl]-urea;
N-[3-((Endo)-8-methyl-8-aza-bicyclo[3.2.1]oct-3-yl)-2,2-diphenyl-propyl]-methanesulfonamide; and/or
(Endo)-3-{2,2-diphenyl-3-[(1-phenyl-methanoyl)-amino]-propyl}-8,8-dimethyl-8-azonia-bicyclo[3.2.1]octane bromide.

Compounds of particular interest that may be useful in combinations of the invention include:
(Endo)-3-(2-methoxy-2,2-di-thiophen-2-yl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane iodide;
(Endo)-3-(2-cyano-2,2-diphenyl-ethyl)-8,8-dimethyl-8-azonia-bicyclo[3.2.1]octane bromide;
(Endo)-3-(2-carbamoyl-2,2-diphenyl-ethyl)-8,8-dimethyl-8-azonia-bicyclo[3.2.1]octane iodide;
(Endo)-3-(2-cyano-2,2-di-thiophen-2-yl-ethyl)-8,8-dimethyl-8-azonia-bicyclo[3.2.1]octane iodide; and/or
(Endo)-3-{2,2-diphenyl-3-[(1-phenyl-methanoyl)-amino]-propyl}-8,8-dimethyl-8-azonia-bicyclo[3.2.1]octane bromide.

Of particular interest is a combination comprising a compound of formula (I) or a pharmaceutically acceptable salt thereof together with an H1 antagonist. Suitable H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. Other histamine receptor antagonists which may be used alone, or in combination with an H3 receptor antagonist include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of formula (I) or a pharmaceutically acceptable salt thereof, together with a PDE4 inhibitor.

The invention thus provides, in a further aspect, a combination comprising a compound of formula (I) or a pharmaceutically acceptable salt thereof, together with a β₂-adrenoreceptor agonist.

The invention thus provides, in a further aspect, a combination comprising a compound of formula (I) or a pharmaceutically acceptable salt thereof, together with an anticholinergic.

The invention thus provides, in a further aspect, a combination comprising a compound of formula (I) or a pharmaceutically acceptable salt thereof, together with a H1 receptor antagonist.

The invention thus provides, in a further aspect, a combination comprising a compound of formula (I) or a pharmaceutically acceptable salt thereof, together with a corticosteroid.

The invention thus provides, in a further aspect, a combination comprising a compound of formula (I) or a pharmaceutically acceptable salt thereof, together with a A2a receptor agonist.

The combinations referred to above may conveniently be presented for use in the form of a composition and thus compositions comprising a combination as defined above, optionally together with a pharmaceutically acceptable diluent or carrier represent a further aspect of the invention.

The individual compounds of such combinations may be administered either sequentially or simultaneously in separate or combined compositions. Suitably, the individual compounds will be administered simultaneously in a combined composition. Appropriate doses of known therapeutic agents will be readily appreciated by those skilled in the art.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The compounds of the invention may be prepared by the methods described below or by similar methods. Thus, the following Descriptions and Examples illustrate the preparation of compounds of the invention. The Examples are not to be considered as limiting the scope of the invention in any way.

### GENERAL EXPERIMENTAL

Throughout the examples, the following abbreviations are used:

AcOH: acetic acid
DCM: dichloromethane

### DMF: N,N-dimethylformamide

EtOAc: ethylacetate
EtOH: ethanol

### LCMS: Liquid Chromatography Mass Spectrometry

MeOH: methanol
mp: melting point
NaOH: sodium hydroxide
PS-DIPEA: polymer supported diisopropylethylamine
RT: retention time
TBTU: O-(benzotriazol-1-yl)-N,N,N′,N′-tetramethyluronium tetrafluoroborate
TFA: trifluoroacetic acid
h: hour(s)
min: minute(s)

SCX cartridges are Ion Exchange SPE columns where the stationary phase is polymeric benzene sulfonic acid. These are used to isolate amines.

SCX2 cartridges are Ion Exchange SPE columns where the stationary phase is polymeric propylsulfonic acid. These are used to isolate amines.

Organic solutions were dried either over magnesium or sodium sulfate.

LCMS was conducted on a Supelcosil LCABZ+PLUS column (3.3 cm×4.6 mm ID) eluting with 0.1% formic acid and 0.01M ammonium acetate in water (solvent A) and 0.05% formic acid 5% water in acetonitrile (solvent B), using the following elution gradient 0.0-7 min 0% B, 0.7-4.2 min 100% B, 4.2-5.3 min 0% B, 5.3-5.5 min 0% B at a flow rate of 3 mlmin⁻¹. The mass spectra were recorded on a Fisons VG Platform spectrometer using electrospray positive and negative mode (ES+ve and ES−ve). Compounds with no symbols were analysed using this method.

In an alternative method, LCMS was conducted on a Waters Atlantis 3μ column (4.6 cm×50 mm ID) eluting with 0.05% formic acid in water (solvent A) and 0.05% formic acid in acetonitrile (solvent B), using the following elution gradient 0.0-4.0 min 0%-100% B at a flow rate of 1 mlmin⁻¹. The mass spectra were recorded on a Waters ZQ platform using electrospray positive and negative mode (ES+ve and ES−ve). Compounds analysed using this method are marked with *.

In yet another method, LCMS was conducted on a Waters Atlantis 3μ column (4.6 cm×50 mm ID) eluting with 0.05% formic acid in water (solvent A) and 0.05% formic acid in acetonitrile (solvent B), using the following elution gradient 0.0-1.5 min 0%-100% B at a flow rate of 1 mlmin⁻¹. The mass spectra were recorded on a Waters ZQ platform using electrospray positive and negative mode (ES+ve and ES−ve). Compounds analysed using this method are marked with ^{ξ}.

The Flashmaster II is an automated multi-user flash chromatography system, available from Argonaut Technologies Ltd, which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength UV-detector and two Gilson FC204 fraction-collectors enabling automated peak cutting, collection and tracking.

Mass directed autopreparative (MDAP) HPLC was conducted on a Waters FractionLynx system comprising of a Waters 600 pump with extended pump heads, Waters 2700 autosampler, Waters 996 diode array and Gilson 202 fraction collector on a 10 cm×2.54 cm id ABZ+ column, eluting with 0.1% formic acid in water (solvent A) and 0.1% formic acid in acetonitrile (solvent B), using as appropriate elution gradient over 15 min at a flow rate of 20 mlmin⁻¹ and detecting at 200-320 nm at room temperature. Mass spectra were recorded on Micromass ZMD mass spectrometer using electro spray positive and negative mode, alternate scans. The software used was MassLynx 3.5 with OpenLynx and FractionLynx options.

Compounds were named using ACD/Name PRO 6.02 chemical naming software Advanced Chemistry Developments Inc.; Toronto, Ontario, M5H2L3, Canada.

### Description 1

### 1,1-Dimethylethyl 4-[(4-iodophenyl)oxy]-1-piperidinecarboxylate (D1)

Sodium hydride (60% oil dispersion, 2.04 g) was added to a solution of N-Boc-4-hydroxypiperidine (commercially available for example from Aldrich) (10 g) in 1-methyl-2-pyrrolidinone (50 ml) under nitrogen at room temperature. 1-Iodo-4-fluorobenzene (commercially available for example from Avocado) (11.1 g) was added at room temperature and then the mixture was heated to 81° C. overnight. The mixture was allowed to cool to room temperature and partitioned between water and EtOAc. The aqueous phase was extracted with more EtOAc and the combined organic solutions were washed with brine, dried and concentrated. The residue was dissolved in DCM and purified by chromatography on Flashmaster II eluting with EtOAc-cyclohexane (0 to 100% over 60 min) to give the title compound (11.6 g) LCMS RT=3.89 min

### Description 2

### Phenylmethyl 4-{4-[(1-{[(1,1-dimethylethyl)oxy]carbonyl}-4-piperidinyl)oxy]phenyl}-3-oxo-1-piperazinecarboxylate (D2)

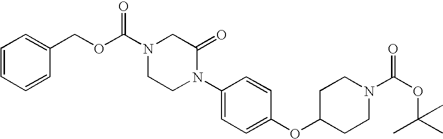

A mixture of 1,1-dimethylethyl 4-[(4-iodophenyl)oxy]-1-piperidinecarboxylate (D1) (10.9 g), 4-benzyloxycarbonylpiperazin-2-one (commercially available for example from Fluorochem) (7.6 g), tripotassium phosphate (6.79 g), N,N′-dimethylethylenediamine (0.29 ml) and copper (I) iodide (257 mg) in dioxane (200 ml) was heated under nitrogen to 100° C. for 24 h. The mixture was allowed to cool to room temperature and partitioned between water and EtOAc. The aqueous solution was extracted with more EtOAc and the combined organic solutions were washed with brine, dried and concentrated. The residue was dissolved in DCM and purified by chromatography on Biotage (400 g) eluting with EtOAc-cyclohexane (2:3 to 1:1) to give the title compound (7.36 g) LCMS RT=3.45 min.

### Description 3

### Phenylmethyl 3-oxo-4-[4-(4-piperidinyloxy)phenyl]-1-piperazinecarboxylate (D3)

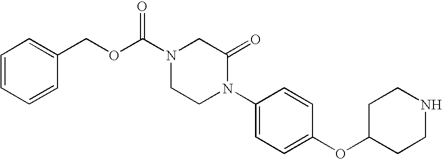

A solution of phenylmethyl 4-{4-[(1-{[(1,1-dimethylethyl)oxy]carbonyl}-4-piperidinyl)oxy]phenyl}-3-oxo-1-piperazinecarboxylate (D2) (7.413 g) in DCM-TFA (4:1; 200 ml) was stirred at room temperature for 1 h and then concentrated under reduced pressure. The residue was diluted with water and basified with NaOH to give a white suspension. The solid was collected by filtration and the aqueous filtrate was extracted with EtOAc. The organic solution was washed with water, brine, dried and evaporated to give the title compound (812 mg). The aqueous solution was basified with more NaOH to pH 13 and extracted with EtOAc. The organic solution was washed with water, brine, dried and evaporated to give additional title compound (2.7 g). The solid (3.5 g) was dissolved in a mixture of DCM and methano (80 ml) and applied to an aminopropyl cartridge (70 g) eluting with MeOH. The appropriate fractions were combined and concentrated to give a further quantity of the title compound (2.28 g). LCMS RT=2.31 min

### Description 4

### Phenylmethyl 4-{4-[(1-cyclobutyl-4-piperidinyl)oxy]phenyl}-3-oxo-1-piperazinecarboxylate (D4)

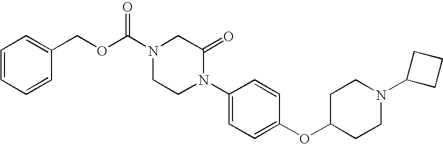

Phenylmethyl 3-oxo-4-[4-(4-piperidinyloxy)phenyl]-1-piperazinecarboxylate (D3) (2.3 g) was dissolved in a mixture of DCM (60 ml), AcOH (3 ml) and cyclobutanone (0.7 ml). The solution was treated portionwise with sodium triacetoxyborohydride (2.37 g) and the mixture was stirred at room temperature under nitrogen for 4 days. The reaction mixture was partitioned between DCM and aqueous sodium hydrogen carbonate. The organic solution was washed with sodium hydrogen carbonate, brine, dried, and concentrated to give the title compound (2.8 g). LCMS RT=2.4 min.

### Description 5

### 1-{4-[(1-Cyclobutyl-4-piperidinyl)oxy]phenyl}-2-piperazinone (D5)

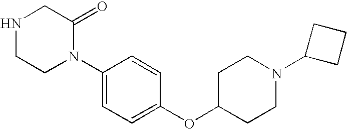

A solution of phenylmethyl 4-{4-[(1-cyclobutyl-4-piperidinyl)oxy]phenyl}-3-oxo-1-piperazinecarboxylate (D4) (2.8 g) in EtOH (30 ml) and EtOAc (50 ml) was hydrogenated over 10% palladium on carbon (400 mg) for 2.5 h. The catalyst was removed by filtration through a pad of celite and washed with EtOH. The combined filtrate and washings were concentrated to give the title compound (1.8 g). LCMS RT=0.46 min

### Description 6

### Phenylmethyl 4-(4-{[1-(1-methylethyl)-4-piperidinyl]oxy}phenyl)-3-oxo-1-piperazinecarboxylate (D6)

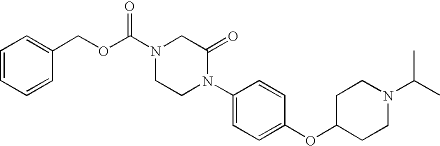

A suspension of phenylmethyl 3-oxo-4-[4-(4-piperidinyloxy)phenyl]-1-piperazinecarboxylate (D3) (545 mg) in acetone (10 ml), DCM (5 ml) and AcOH (1 ml) was treated portionwise with sodium triacetoxyborohydride (0.57 g) and the mixture was stirred at room temperature under nitrogen overnight. A further portion of sodium triacetoxyborohydride (0.57 g) was added, and the mixture was stirred for a further day. The reaction mixture was partitioned between EtOAc and aqueous sodium hydrogen carbonate. The organic solution was washed with sodium hydrogen carbonate, brine, dried, and concentrated to give the title compound (576 mg). LCMS RT=2.36 min.

### Description 7

### 1-(4-{[1-(1-Methylethyl)-4-piperidinyl]oxy}phenyl)-2-piperazinone (D7)

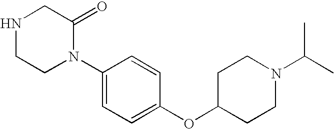

A solution of phenylmethyl 4-(4-{[1-(1-methylethyl)-4-piperidinyl]oxy}phenyl)-3-oxo-1-piperazinecarboxylate (D6) (1.168 g) in EtOH (20 ml) and EtOAc (5 ml) was hydrogenated over 10% Palladium on carbon (200 mg) for 2 h. More catalyst (200 mg) was added and the mixture was stirred under hydrogen for a further 1.5 h. The catalyst was removed by filtration and the filtrate was hydrogenated over a fresh batch of catalyst (200 mg) overnight. The catalyst was removed by filtration through a celite cartridge (10 g) and washed with EtOH. The combined filtrate and washings were concentrated to give the title compound (732 mg). LCMS RT=0.29 min

### Description 8

### Phenylmethyl 4-{4-[(1-cyclopentyl-4-piperidinyl)oxy]phenyl}-3-oxo-1-piperazinecarboxylate (D8)

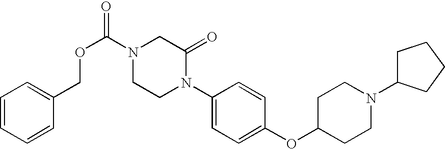

Phenylmethyl 3-oxo-4-[4-(4-piperidinyloxy)phenyl]-1-piperazinecarboxylate (D3) (2.3 g) was dissolved in DCM (60 ml), AcOH (3 ml) and cyclopentanone (0.7 ml). The solution was treated portionwise with sodium triacetoxyborohydride (2.37 g) and the mixture was stirred at room temperature under nitrogen overnight. A further portion of sodium triacetoxyborohydride (2.37 g) was added and the mixture was stirred for a further 3 days. The reaction mixture was partitioned between DCM and aqueous sodium hydrogen carbonate. The organic solution was washed with sodium hydrogen carbonate, brine, dried, and concentrated to give the title compound (2.7 g). LCMS RT=2.5 min.

### Description 9

### 1-{4-[(1-Cyclopentyl-4-piperidinyl)oxy]phenyl}-2-piperazinone (D9)

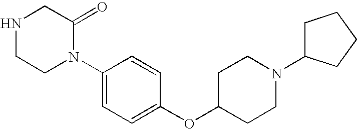

A solution of phenylmethyl 4-{4-[(1-cyclopentyl-4-piperidinyl)oxy]phenyl}-3-oxo-1-piperazinecarboxylate (D8) (2.8 g) in EtOH (30 ml) and EtOAc (50 ml) was hydrogenated over 10% palladium on carbon (400 mg) for 2.5 h. The catalyst was removed by filtration through a pad of celite and washed with EtOH. The combined filtrate and washings were concentrated to give the title compound (1.88 g). LCMS RT=0.6 min.

### Description 10

### N²-(2-Hydroxyethyl)-N¹-{4-[(phenylmethyl)oxy]phenyl}glycinamide (D10)

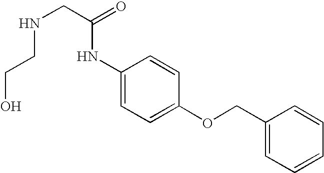

4-[(Phenylmethyl)oxy]aniline (10 g) was stirred in dry tetrahydrofuran (100 ml), and cooled in an ice/water bath to ˜5° C. A solution of potassium carbonate (16.15 g) in water (60 ml) was added to the above mixture, followed by the dropwise addition of chloroacetyl chloride (4.22 ml) over 30 min. The mixture was allowed to warm up to room temperature and the organic phase separated. The organic phase was cooled to ˜5° C. in an ice/water bath and 2-aminoethanol (9 g) added. The mixture was allowed to warm up to room temperature and heated at 60° C. for 2 h, left overnight at room temperature and heated at 60° C. for a further 2 h. The reaction mixture was partitioned between EtOAc and water. The organic phase was separated, washed with water, brine, dried and concentrated. The residue was recrystallised from chloroform to give the title compound (8.1 g). LCMS RT=2.17 min.

### Description 11

### 1,1-Dimethylethyl (2-hydroxyethyl)[2-oxo-2-({4-[(phenylmethyl)oxy]phenyl}amino)ethyl]carbamate (D11)

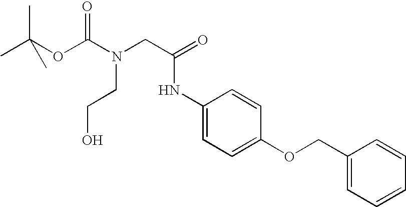

Bis(1,1-dimethylethyl)dicarbonate (14.5 g), 4-dimethylaminopyridine (150 mg) were added to a solution of N²-(2-hydroxyethyl)-N′-{4-[(phenylmethyl)oxy]phenyl}glycinamide (D10) (8.1 g) in DCM (200 ml). The mixture was stirred for 30 min, and aqueous saturated sodium bicarbonate added (150 ml). The organic phase was separated, dried and evaporated. The residue was dissolved in MeOH (150 ml) and potassium carbonate (14.5 g) added. The mixture was heated on a steam bath for 15 min, allowed to cool to room temperature and left standing for 18 h. The reaction mixture was evaporated and partitioned between DCM and water. The organic phase was separated, washed with brine, dried and evaporated. The residue was pre-absorbed onto flash silica and purified by flash chromatography, (100 g silica cartridge; 0% to 100% EtOAc-cyclohexane gradient over 30 min), to give the title compound (5.0 g). LCMS RT=3.2 min.

### Description 12

### 1,1-Dimethylethyl 3-oxo-4-{4-[(phenylmethyl)oxy]phenyl}-1-piperazinecarboxylate (D12)

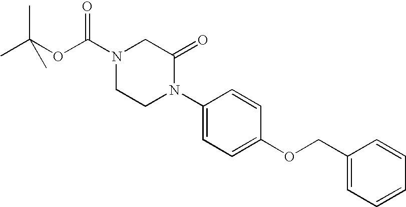

Methanesulfonyl chloride (2.5 ml) was added to a solution of 1,1-dimethylethyl (2-hydroxyethyl)[2-oxo-2-({4-[(phenylmethyl)oxy]phenyl}amino)ethyl]carbamate (D11) (4.99 g) in dry DCM (50 ml) and triethylamine (20 ml) under nitrogen. The mixture was stirred for 30 min, and then partitioned between DCM and aqueous saturated sodium bicarbonate. The organic layer was separated and concentrated in vacuo. The residue was dissolved in dry DMF (20 ml) and sodium hydride (60% oil dispersion, 0.6 g) added. The mixture was stirred for 30 min, quenched with aqueous saturated sodium bicarbonate and extracted with EtOAc. The organic phases were combined, dried and evaporated. The residue was purified by flash chromatography, (100 g silica cartridge; 0% to 100% EtOAc-cyclohexane gradient over 40 min), to give the title compound (4.1 g). LCMS RT=3.2 min.

### Description 13

### 1,1-Dimethylethyl 4-(4-hydroxyphenyl)-3-oxo-1-piperazinecarboxylate (D13)

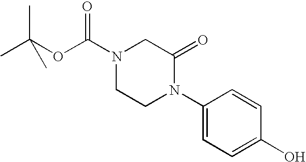

A solution of 1,1-dimethylethyl 3-oxo-4-{4-[(phenylmethyl)oxy]phenyl}-1-piperazinecarboxylate (D12) (3.1 g) in EtOH (25 ml) and EtOAc (25 ml) was hydrogenated over 10% palladium on carbon (1 g) for 2 h. The catalyst was removed by filtration through celite and washed with EtOH. The combined filtrate and washings were concentrated to give the title compound (1.92 g). LCMS RT=2.5 min

### Description 14

### 1,1-Dimethylethyl 4-{4-[(3-chloropropyl)oxy]phenyl}-3-oxo-1-piperazinecarboxylate (D14)

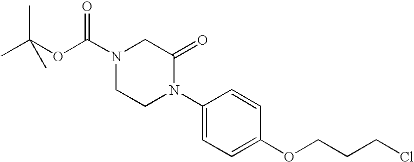

To a solution of 1,1-dimethylethyl 4-(4-hydroxyphenyl)-3-oxo-1-piperazinecarboxylate (D13) (5.0 g) in 2-butanone (200 ml) was added potassium carbonate (4.7 g) followed by 1-bromo-3-chloropropane (4.0 g). The mixture was heated at 90° C. under nitrogen for 72 h, cooled to room temperature and water (200 ml) added. The organic phase was separated, and the aqueous layer extracted twice with DCM. The organic phases were combined, dried and evaporated. The residue was purified by flash chromatography, (100 g silica cartridge; 0% to 100% EtOAc-cyclohexane gradient over 60 min), to give a mixture of the title compound and 1,1-dimethylethyl 4-{4-[(3-bromopropyl)oxy]phenyl}-3-oxo-1-piperazinecarboxylate (6.2 g; 9:1). LCMS RT=3.2 min.

### Description 15

### 1,1-Dimethylethyl 3-oxo-4-(4-{[3-(1-pyrrolidinyl)propyl]oxy}phenyl)-1-piperazinecarboxylate (D15)

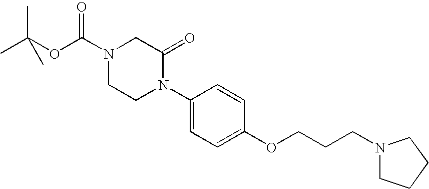

A solution of 1,1-dimethylethyl 4-{4-[(3-chloropropyl)oxy]phenyl}-3-oxo-1-piperazinecarboxylate (D14) (0.87 g) was dissolved in 2-butanone (40 ml). Potassium iodide (0.110 g) and pyrrolidine (0.387 ml) were added. The mixture was heated at 80° C. overnight. The reaction mixture was cooled and more pyrrolidine (0.386 ml) followed by potassium carbonate (0.635 g) were added. The reaction mixture was heated at 85° C. over the weekend. The reaction mixture was allowed to cool to room temperature and then concentrated in vacuo. The residue was partitioned between EtOAc and saturated sodium bicarbonate solution. The aqueous was extracted with EtOAc and the combined organics washed with water, brine, dried and evaporated. The residue was purified by Biotage column chromatography (90 g cartridge) eluting with DCM-EtOH-aqueous ammonia solution (200:8:1), increasing to 100:8:1, to give the title compound (578 mg). LCMS RT=2.08 min.

### Description 16

### 1-(4-{[3-(1-Pyrrolidinyl)propyl]oxy}phenyl)-2-piperazinone bis(trifluoroacetate) (D16)

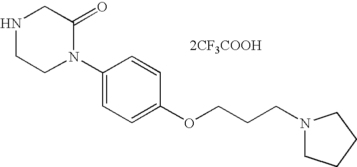

A solution of 1,1-dimethylethyl 3-oxo-4-(4-{[3-(1-pyrrolidinyl)propyl]oxy}phenyl)-1-piperazinecarboxylate (D15) (578 mg) in DCM-TFA (20 ml, 4:1) was stirred at room temperature for 45 min. The solvents were removed in vacuo to leave the title compound (1.2 g). LCMS RT=0.34 min.

### Description 17

### 1,1-Dimethylethyl 4-(4-{[3-(2-methyl-1-pyrrolidinyl)propyl]oxy}phenyl)-3-oxo-1-piperazinecarboxylate (D17)

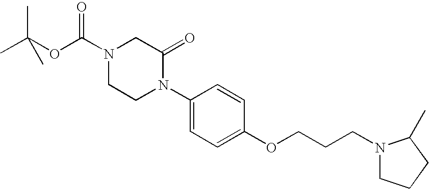

A solution of 1,1-dimethylethyl 4-{4-[(3-chloropropyl)oxy]phenyl}-3-oxo-1-piperazinecarboxylate (D14) (1.3 g) was dissolved in 2-butanone (50 ml). Potassium carbonate (0.96 g), potassium iodide (1.16 g) and 2-methylpyrrolidine (commercially available for example from Fisher) (1.5 g) were added. The mixture was heated at 80° C. for 24 h. The reaction mixture was cooled to room temperature and partitioned between EtOAc and water. The organic phase was dried and concentrated. The residue was purified by flash chromatography, [100 g silica cartridge; 0% to 15% (10% 0.880 ammonia in MeOH)— DCM gradient over 30 min]. The appropriate fractions were combined and concentrated to give the title compound (0.85 g). LCMS RT=2.28 min.

### Description 18

### 1-(4-{[3-(2-Methyl-1-pyrrolidinyl)propyl]oxy}phenyl)-2-piperazinone (D18)

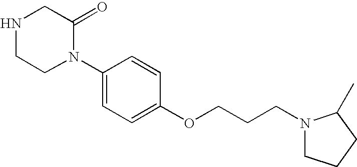

1,1-Dimethylethyl 4-[4-({3-[(2-methyl-1-pyrrolidinyl]propyl}oxy)phenyl]-3-oxo-1-piperazinecarboxylate (D17) (0.85 g) was dissolved in DCM (10 ml) and TFA-DCM mixture (10 ml, 2:5) added. The reaction mixture was stirred at room temperature for 18 h. The solvent was removed in vacuo. The residue was dissolved in MeOH and the solution was applied to an SCX-2 cartridge (70 g). The cartridge was washed with MeOH and the product eluted with 10% 0.880 ammonia in MeOH. The appropriate ammoniacal fractions were combined and concentrated to give the title compound (645 mg). LCMS RT=0.36 min.

### Description 19

### 1,1-Dimethylethyl 4-[4-({3-[(2S)-2-methyl-1-pyrrolidinyl]propyl}oxy)phenyl]-3-oxo-1-piperazinecarboxylate (D19)

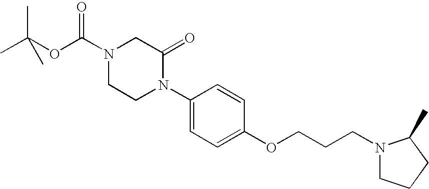

A solution of 1,1-dimethylethyl 4-{4-[(3-chloropropyl)oxy]phenyl}-3-oxo-1-piperazinecarboxylate (D14) (0.74 g) was dissolved in 2-butanone (25 ml). Potassium carbonate (0.55 g), potassium iodide (0.66 g) and (2S)-2-methylpyrrolidine hydrochloride (which can be prepared as described in US 20040171845) (0.2 g) were added. The mixture was heated at 80° C. overnight. The reaction mixture was cooled to room temperature and partitioned between DCM and water. The organic phase was dried and concentrated. The residue was purified first by flash chromatography, [100 g silica cartridge; 0% to 30% (1% triethylamine in MeOH)-DCM gradient over 60 min] and then by applying to a 20 g SCX-2 ion exchange cartridge in MeOH and eluting with 10% 0.880 ammonia in MeOH solution. The appropriate ammoniacal fractions were combined and concentrated to give the title compound (0.418 g). LCMS RT=2.2 min.

### Description 20

### 1-[4-({3-[(2S)-2-Methyl-1-pyrrolidinyl]propyl}oxy)phenyl]-2-piperazinone (D20)

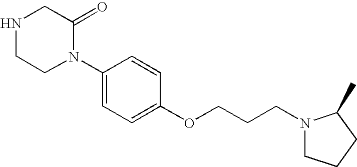

1,1-Dimethylethyl 4-[4-({3-[(2S)-2-methyl-1-pyrrolidinyl]propyl}oxy)phenyl]-3-oxo-1-piperazinecarboxylate (D19) (0.418 g) was dissolved in DCM (10 ml) and TFA (2 ml) added. The reaction mixture was stirred at room temperature for 2-3 h. The solvent was removed in vacuo to leave the title compound (0.32 g). LCMS RT=0.37 min.

### Description 21

### 1,1-Dimethylethyl 3-oxo-4-(4-{[3-(1-piperidinyl)propyl]oxy}phenyl)-1-piperazinecarboxylate (D21)

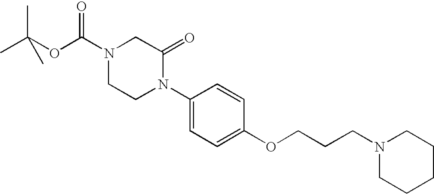

To a solution of 1,1-dimethylethyl 4-{4-[(3-chloropropyl)oxy]phenyl}-3-oxo-1-piperazinecarboxylate (D14) (1.35 g) in acetone (50 ml) was added sodium iodide (0.517 g) and piperidine (2 ml). The resultant mixture was heated to 60° C. and stirred for 16 h and then at room temperature for 5 days. More sodium iodide (0.517 g) and piperidine (2 ml) were added and the reaction mixture was stirred at 60° C. for a further 5 h. The reaction mixture was allowed to cool to room temperature and concentrated in vacuo. The residue was partitioned between EtOAc and saturated sodium bicarbonate solution. The aqueous was extracted with EtOAc and the combined organic solutions were washed with water, brine, dried (Na₂SO₄) and evaporated. The residue was purified by Biotage column chromatography (90 g cartridge) and eluted with DCM-EtOH-aqueous ammonia (150:8:1) to give the title compound (0.345 g). LCMS RT=2.20 min.

### Description 22

### 1-(4-{[3-(1-Piperidinyl)propyl]oxy}phenyl)-2-piperazinone bis(trifluoroacetate) (D22)

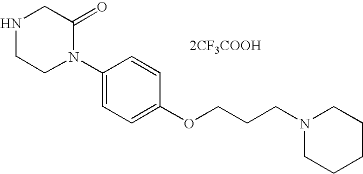

A solution of 1,1-dimethylethyl 3-oxo-4-(4-{[3-(1-piperidinyl)propyl]oxy}phenyl)-1-piperazinecarboxylate (D21) (0.345 g) in DCM-TFA (20 ml, 4:1) was stirred at room temperature for 45 min. The solvents were then removed in vacuo to leave the title compound (0.745 g). LCMS RT=0.27 min

### Description 23

### 1,1-Dimethylethyl 4-(4-{[3-(hexahydro-1H-azepin-1-yl)propyl]oxy}phenyl)-3-oxo-1-piperazinecarboxylate (D23)

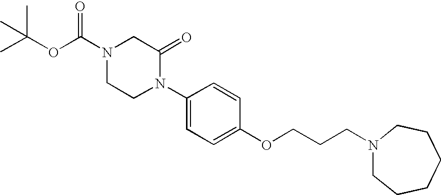

A solution of 1,1-dimethylethyl 4-{4-[(3-chloropropyl)oxy]phenyl}-3-oxo-1-piperazinecarboxylate (D14) (1.3 g) was dissolved in 2-butanone (50 ml). Potassium carbonate (0.96 g), potassium iodide (1.16 g) and hexahydro-1H-azepine (1.8 g) were added. The mixture was heated at 80° C. for 24 h. The reaction mixture was cooled to room temperature and partitioned between EtOAc and water. The organic phase was dried and concentrated. The residue was purified by flash chromatography [100 g silica cartridge; 0% to 15% (10% 0.880 ammonia in MeOH)-DCM gradient over 30 min]. The appropriate fractions were combined and concentrated to give the title compound (0.9 g). LCMS RT=2.35 min.

### Description 24

### 1-(4-{[3-(Hexahydro-1H-azepin-1-yl)propyl]oxy}phenyl)-2-piperazinone (D24)

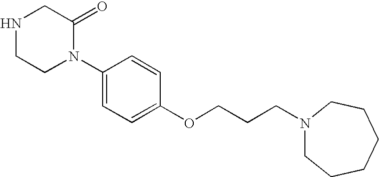

1,1-Dimethylethyl 4-(4-{[3-(hexahydro-1H-azepin-1-yl)propyl]oxy}phenyl)-3-oxo-1-piperazinecarboxylate (D23) (0.9 g) was dissolved in DCM (10 ml) and TFA-DCM mixture (10 ml, 2:5) added. The reaction mixture was stirred at room temperature for 18 h. The solvent was removed in vacuo. The residue was dissolved in MeOH and the solution was applied to an SCX-2 cartridge (70 g). The cartridge was washed with MeOH and the product eluted with 10% 0.880 ammonia in MeOH. The appropriate ammoniacal fractions were combined and concentrated to give the title compound (0.67 g). LCMS RT=0.54 min.

### Description 25

### 1,1-Dimethylethyl 4-[4-({3-[(2R)-2-methyl-1-pyrrolidinyl]propyl}oxy)phenyl]-3-oxo-1-piperazinecarboxylate (D25)

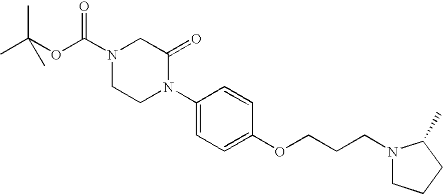

A mixture of 1,1-dimethylethyl 4-{4-[(3-chloropropyl)oxy]phenyl}-3-oxo-1-piperazinecarboxylate and 1,1-dimethylethyl 4-{4-[(3-bromopropyl)oxy]phenyl}-3-oxo-1-piperazinecarboxylate (D14) (9:1; 2.58 g) was dissolved in 2-butanone (50 ml). Potassium carbonate (2.9 g), potassium iodide (2.32 g) and (2R)-2-methylpyrrolidine hydrochloride (which can be prepared as described in US20040171845) (1.0 g) were added. The mixture was heated at 90° C. under nitrogen for 72 h. The reaction mixture was cooled to room temperature and partitioned between DCM and water. The organic phase was dried and concentrated. The residue was purified by flash chromatography, [100 g silica cartridge; 0% to 30% (1% triethylamine/MeOH)-DCM gradient over 60 min], to give the title compound (1.87 g). LCMS RT=2.2 min.

### Description 26

### 1-[4-({3-[(2R)-2-Methyl-1-pyrrolidinyl]propyl}oxy)phenyl]-2-piperazinone (D26)

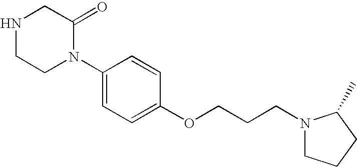

A solution of TFA (4 ml) in DCM (6 ml) was added to 1,1-dimethylethyl 4-[4-({3-[(2R)-2-methyl-1-pyrrolidinyl]propyl}oxy)phenyl]-3-oxo-1-piperazinecarboxylate (D25) (1.87 g) in DCM (10 ml). The mixture was stirred under nitrogen for 2 h. TFA (4 ml) was added and the mixture stirred for a further 18 h. The mixture was evaporated and the residue dissolved in MeOH. The solution was applied to an SCX-2 cartridge (70 g). The cartridge was washed with MeOH and the product eluted with 10% 0.880 ammonia in MeOH. The appropriate ammoniacal fractions were combined and concentrated to give the title compound (1.39 g). LCMS RT=0.35 min.

### Description 27

### Methyl 4-({[2-(methyloxy)ethyl]amino}carbonyl)benzoate (D27)

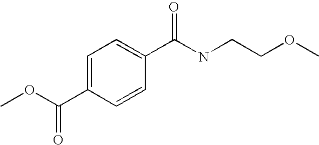

A solution of methyl 4-(chlorocarbonyl)benzoate (commercially available for example from Acros) (1.09 g) in DCM (5 ml) was treated with triethylamine (2 ml), followed by 2-methoxyethylamine (0.87 ml) and diluted with more DCM (5 ml). The reaction mixture was allowed to react for 2 h at room temperature with occasional shaking. The solvent was removed under reduced pressure and the residue partitioned between EtOAc and 2M hydrochloric acid. The organic solution was washed with brine, dried, and evaporated to give the title compound (1.06 g). LCMS RT=2.26 min

### Description 28

### Methyl 4-({methyl[2-(methyloxy)ethyl]amino}carbonyl)benzoate (D28)

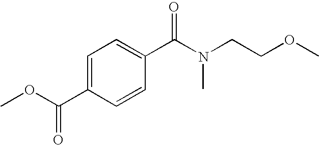

A solution of methyl 4-({[2-(methyloxy)ethyl]amino}carbonyl)benzoate (D27) (210 mg) in DMF (2 ml) was treated with sodium hydride (60% oil dispersion, 98 mg) under nitrogen. After 10 min, methyliodide (1 ml) was added and the mixture was stirred at room temperature overnight. The reaction mixture was partitioned between EtOAc and 2M hydrochloric acid. The organic solution was washed with aqueous sodium bicarbonate, brine, dried and evaporated to dryness. The residue was chromatographed on a 20 g silica cartridge eluting with 0 to 100% EtOAc-cyclohexane over 40 min on Flashmaster to give the title compound (241 mg). LCMS RT=2.33 min.

### Description 29

### 4-({Methyl[2-(methyloxy)ethyl]amino}carbonyl)benzoic acid (D29)

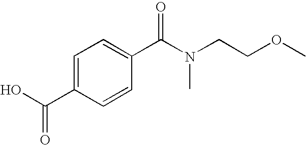

A solution of methyl 4-({methyl[2-(methyloxy)ethyl]amino}carbonyl)benzoate (D28) (0.24 g) in MeOH (10 ml) was treated with 2M NaOH (5 ml) and the mixture was heated to 40° C. for 2 h. The mixture was concentrated under reduced pressure and the residue was suspended in 2M hydrochloric acid and stirred for 10 min and then EtOAc was added. The organic solution was washed with 2M hydrochloric acid, brine, dried and evaporated. The residue was treated with diethyl ether and re-evaporated to give the title compound (140 mg) LCMS RT=2.05 min.

### Description 30

### 1-(3,4-Dihydro-2H-1-benzothiopyran-6-yl)ethanone (D30)

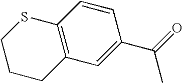

A solution of aluminium chloride (5.86 g) in DCM (20 mL) was treated with a solution of acetic anhydride (2.24 g) in DCM (10 mL). The solution was cooled in an ice-water cooling-bath and then treated dropwise with a solution of 3,4-dihydro-2H-1-benzothiopyran (3.11 g) in DCM (15 mL). The mixture was stirred for 1 h with ice-cooling, and then was poured into ice-water. The organic layer was washed with water and saturated sodium bicarbonate solution, dried, filtered, evaporated under reduced pressure and the residue was recrystallised from benzene-petroleum ether to give the title compound (1.86 g) mp 49.5-50.5° C.

### Description 31

### 3,4-Dihydro-2H-1-benzothiopyran-6-carboxylic acid 1,1-dioxide (D31)

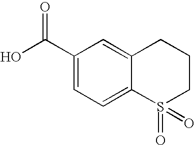

A mixture of 1-(3,4-dihydro-2H-1-benzothiopyran-6-yl)ethanone (6.68 g) and sodium hypochlorite solution (7.2%, 140 mL) was heated to 70° C. for 4 h. The mixture was cooled and extracted with chloroform. The aqueous layer was treated with sodium metabisulfite (30 g) until no chlorine gas was evolved on acidification. The mixture was acidified, and the solid was collected by filtration. The solid was washed with water, dried and recrystallised from DCM to give the title compound (2.46 g) mp 246-249° C.

### Description 32

### Methyl 4-(azetidin-1-ylcarbonyl)benzoate (D32)

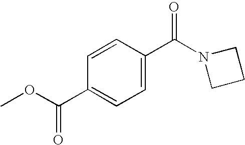

Methyl 4-chlorocarbonylbenzoate (2.0 g) was stirred with triethylamine (2.0 ml) in DCM (20 ml) at 20° C. and azetidine (0.81 ml) was added. (It is to be noted that this reaction was vigorous, and thus cooling and slower addition of azetidine may be advisable) After stirring at room temperature under nitrogen for 2 h, the solution was diluted with DCM and washed with water twice. The organic solution was then washed with dilute hydrochloric acid (2M) and water, each time back extracting with DCM. The combined organic layers were dried with magnesium sulphate and evaporated to give the title compound (1.92 g), LCMS RT=2.33 min. ES+ve m/z 220 (M+H)⁺.

### Description 33

### 4-(Azetidin-1-ylcarbonyl)benzoic acid (D33)

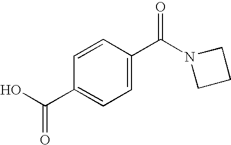

Methyl 4-(azetidin-1-ylcarbonyl)benzoate (D32) (1.91 g) was heated at 70° C. in MeOH (20 ml) and 2M NaOH solution (10 ml) with stirring for 1.5 h. After cooling, the mixture was acidified with 2M hydrochloric acid (10 ml) to pH 5. The white solid which separated was collected by filtration and washed with water. The combined filtrate and washings were acidified by adding further 2M hydrochloric acid to pH 1 and the solution was extracted three times with EtOAc. The combined organic solutions were washed with brine, dried and evaporated to give the title compound (0.25 g), LCMS RT=2.04 min.

### Description 34

### 4-(1-Pyrrolidinylcarbonyl)benzoic acid (D34)

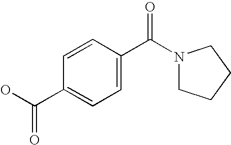

Methyl 4-chlorocarbonylbenzoate (2.33 g) was stirred with triethylamine (2.0 ml) in DCM (12 ml) at 20° C. and pyrrolidine (1.5 ml) was added. After 2.5 h, the solution was concentrated under reduced pressure and the residue was partitioned between EtOAc and 2M hydrochloric acid. The organic solution was then washed with dilute hydrochloric acid, brine, dried and evaporated. The residue was dissolved in MeOH (5 ml) and treated with 2M NaOH (10 ml) and the mixture was heated to reflux for 2 h. The solvent was removed under reduced pressure and the residue was suspended in 2M hydrochloric acid and stirred for 10 min. The solid was collected by filtration, washed with 2M hydrochloric acid and air dried. This was purified by chromatography on a silica 100 g cartridge eluting with 5 to 20% MeOH in DCM over 40 min on Flashmaster to give the title compound (535 mg), LCMS RT=2.19 min.

### Description 35

### 3-(1-Pyrrolidinylcarbonyl)benzoic acid (D35)

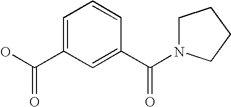

A solution of methyl 3-(1-pyrrolidinylcarbonyl)benzoate (which can be prepared as described in WO2003004468) (2.23 g) in EtOH (10 ml) was heated with 2M NaOH for 15 h at 80° C. The solvent was removed under reduced pressure and the residue was acidified with 2M hydrochloric acid and extracted with EtOAc. The organic solution was dried and evaporated to give the title compound (1.35 g) LCMS RT=2.11 min.

The following carboxylic acids are commercially available from Aldrich:

pivalic acid, cyclobutanecarboxylic acid, cyclopentanecarboxylic acid, 4-ethylbenzoic acid, benzoic acid, 4-chloro-2-(methyloxy)benzoic acid, 4-methyl-3-(methyloxy)benzoic acid, 4-fluorobenzoic acid, 4-chlorobenzoic acid, 4-methylbenzoic acid, cyclohexanecarboxylic acid, 1-methylcyclohexanecarboxylic acid, 3,3-dimethylbutanoic acid, 1-naphthalenecarboxylic acid, 4-pyridinecarboxylic acid, 2,4-difluorobenzoic acid, 1,3-benzothiazole-6-carboxylic acid, 3-pyridinecarboxylic acid, 4-(methylsulfonyl)benzoic acid, 2-methyl-3-pyridinecarboxylic acid, 3,5-difluorobenzoic acid, 6-methyl-3-pyridinecarboxylic acid, 2,3-difluorobenzoic acid, 2,5-difluorobenzoic acid, 2,6-difluorobenzoic acid, 3,4-difluorobenzoic acid, 3,4-dichlorobenzoic acid, 3-cyanobenzoic acid, 4-bromobenzoic acid, 4-cyanobenzoic acid, 1,3-benzodioxole-5-carboxylic acid, cyclopropanecarboxylic acid.

3-Chloro-4-(methyloxy)benzoic acid, 6-quinolinecarboxylic acid are available from Avocado.

1,2-Dimethyl-1H-indole-3-carboxylic acid, 8-fluoro-5-quinolinecarboxylic acid, 4-(2-amino-2-oxoethyl)benzoic acid are available from Peakdale Molecular Ltd.

1-Methyl-1H-1,2,3-benzotriazole-5-carboxylic acid, 4-(1,3-oxazol-5-yl)benzoic acid are available from Maybridge Chemical Intermediates.

The following compounds are available from Acros: 3-(methylsulfonyl)benzoic acid, 1,5-dimethyl-1H-pyrazole-3-carboxylic acid, 3,5-dimethyl-4-isoxazolecarboxylic acid.

5-Quinolinecarboxylic acid is available from Lancaster.

1-Methyl-5-oxo-3-pyrrolidinecarboxylic acid, 3-(1-methylethyl)-4-(methyloxy)benzoic acid are available from Sigma Aldrich Lib Rare Chemicals (Salor).

Pyrazolo[1,5-a]pyrimidine-3-carboxylic acid is available from ChemBridge Corporation.

4-{[2-(Methyloxy)-2-oxoethyl]oxy}-1-naphthalenecarboxylic acid may be prepared according to the methods described in WO2004035556.

4-Cyano-2-(methyloxy)benzoic acid may be prepared according to the procedure in EP0166609.

3-Cyano-4-(methyloxy)benzoic acid may be prepared according to the procedure in WO2004056369.

4-(4-Morpholinylcarbonyl)benzoic acid may be prepared according to the procedure in J. Med. Chem., 37:4538-53, (1994).

The exemplified compounds E33 to E168 were prepared following the general acylation method described below or similar method:

Thus, a mixture of the appropriate carboxylic acid listed in the table (0.1 mmol), the appropriate amine listed in the table (0.1 mmol), with a suitable base such as triethylamine (1 mmol) or diisopropylethylamine (0.2 mmol) in a solvent such as dimethylformamide (0.5 ml) or dichloromethane (1 mL) was treated with an appropriate activating agent such as TBTU (0.15 mmol) or O-(7-azabenzotriazol-1-yl)-N,N,N′,N′-tetramethyluronium hexa-fluorophosphate (HATU) (0.1 mmol) and the mixture was stirred for a period of time between 3 h and overnight at room temperature. Optionally, the reaction mixture was washed with water and/or brine as appropriate and dried (MgSO₄). The reaction mixture was then concentrated under reduced pressure and the residue was dissolved in MeOH. The solution was applied to a 10 g SCX-2 ion-exchange cartridge which was pre-conditioned with MeOH. The cartridge was washed with MeOH and the product eluted with 10% aqueous ammonia in MeOH. The ammoniacal fractions were combined and evaporated under reduced pressure. The residue was purified by mass-directed auto-preparative HPLC to give the compounds listed in the table below. In a second purification method, the residue was purified by flash chromatography on silica, eluting with 2-5% (10% ammonia/MeOH) in dichloromethane.

It is believed that compounds E1-E31 and E169-175 can be prepared using the above general acylation procedure from the appropriate carboxycylic acid and amine given in the table below.

In an alternative preparation, the compounds of the invention may be prepared from the appropriate acid chloride listed in the table and the appropriate amines listed in the table under standard coupling conditions well-known to those skilled in the art. Specific compounds were prepared according to the methods described below.

### EXAMPLES 1 TO 5

A mixture of the appropriate acid chloride listed in the table (0.435 mmol), the appropriate amine listed in the table (0.395 mmol), and triethylamine (0.435 mmol) in dichloromethane (3 ml) was stirred overnight. The reaction mixture was washed with water, dried over sodium sulphate and evaporated. Purified by chromatography on silica, eluting with 5% (10% ammonia/MeOH) dichloromethane. The products were converted to the HCl salts by dissolving in dichloromethane, adding 1 equivalent of 1M HCl in diethyl ether, evaporating and drying to afford the compounds listed below.

### EXAMPLE 27 AND 28

A mixture of the appropriate acid chloride listed in the table (0.52 mmol), the appropriate amine listed in the table (0.47 mmol), and triethylamine (0.52 mmol) in dichloromethane (3 ml) was stirred overnight. The reaction mixture was stirred with a saturated solution of sodium bicarbonate for 30 minutes, then washed with water, dried over sodium sulphate and evaporated. They were purified by chromatography on silica. The products were converted to the HCl salts by dissolving in dichloromethane, adding an excess of 1M HCl in diethyl ether, evaporating and drying to afford the compounds listed below.

### EXAMPLE 175

A suspension of the amine (D22) (0.922 mmol) in dichloromethane was treated with pyridine (3.228 mmol) followed by the appropriate acid chloride (1.014 mmol) listed in the table and stirred overnight. The solvent was evaporated and the residue diluted with a saturated solution of sodium bicarbonate and the solution stirred for 30 minutes. Extraction into organic solvent failed so the mixture was acidified using acetic acid and passed through a SCX cartridge, eluting sequentially with water/MeOH/ammonia/ethanol. Purified using chromatography on silica, eluting with 5% (10% ammonia/MeOH) dichloromethane. The products were converted to the HCl salts by dissolving in dichloromethane, adding 1 equivalent of 1M HCl in diethyl ether, evaporating and drying to afford the compound listed below.

### EXAMPLES 6, 14 AND 15

The amine (D22) (0.41 mmol) was dissolved in dichloromethane (5 ml), treated with diethylaminomethyl polystyrene (3.2 mmol/g) (0.62 mmol) and the appropriate acid chloride listed in the table (0.62 mmol) and left to stir at room temperature under argon for 30 minutes. The mixture was diluted with MeOH and passed down an SCX column, eluting with MeOH, followed by 10% ammonia/MeOH. The basic fractions were combined and evaporated under reduced pressure to yield the compounds listed below.

| Example | | | | RT | |
|---|---|---|---|---|---|
| number | Structure | Amine | Acid or acid chloride | (min) | m/z |
| E1 | 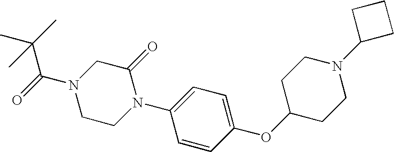 | D5 | pivalic acid or 2,2-dimethylpropanoylchloride | 1.53* | 414 |
| E2 | 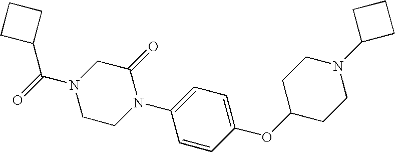 | D5 | cyclobutanecarboxylicacid orcyclobutanecarbonylchloride | 1.46* | 412 |
| E3 | 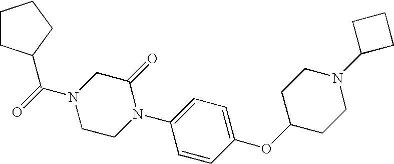 | D5 | cyclopentanecarboxylicacid orcyclopentanecarbonylchloride | 1.58* | 426 |
| E4 | 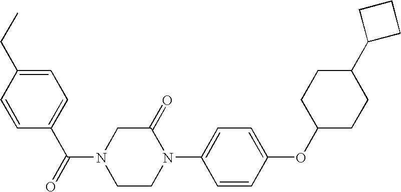 | D5 | 4-ethylbenzoic acid or 4-ethylbenzoyl chloride | 1.78* | 462 |
| E5 | 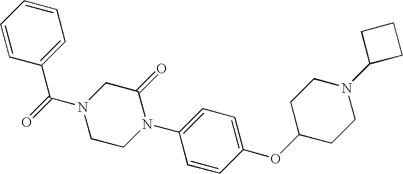 | D5 | benzoic acid or benzoylchloride | 1.51* | 434 |
| E6 | 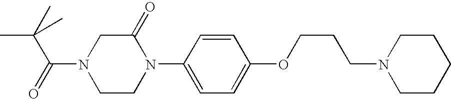 | D22 | pivalic acid or 2,2-dimethylpropanoylchloride | 1.69* | 402 |
| E7 | 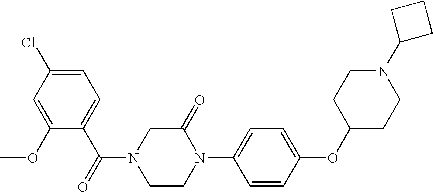 | D5 | 4-chloro-2-(methyloxy)benzoic acid | 0.77^{ξ} | 498,500 |
| E8 | 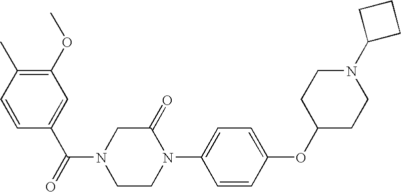 | D5 | 4-methyl-3-(methyloxy)benzoic acid | 0.78^{ξ} | 478 |
| E9 | 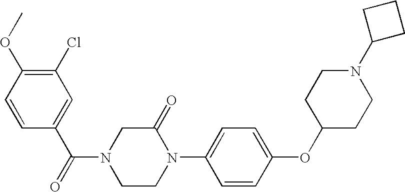 | D5 | 3-chloro-4-(methyloxy)benzoic acid | 0.77^{ξ} | 498,500 |
| E10 | 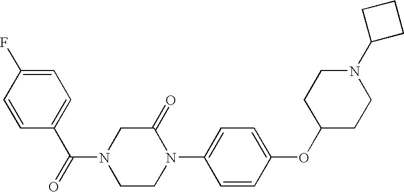 | D5 | 4-fluorobenzoic acid | 0.70^{ξ} | 452 |
| E11 | 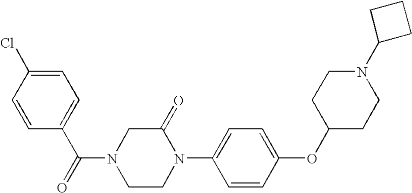 | D5 | 4-chlorobenzoic acid | 0.77^{ξ} | 468,470 |
| E12 | 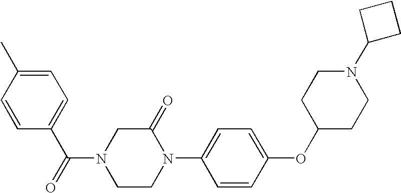 | D5 | 4-methylbenzoic acid | 0.75^{ξ} | 448 |
| E13 | 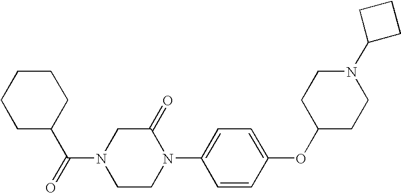 | D5 | cyclohexanecarboxylicacid | 0.75^{ξ} | 440 |
| E14 | 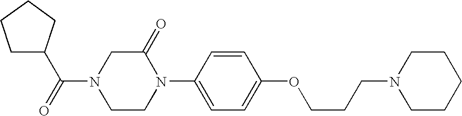 | D22 | cyclopentanecarboxylicacid orcyclopentanecarbonylchloride | 1.73^{ξ} | 414 |
| E15 | 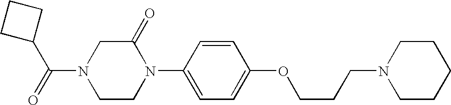 | D22 | cyclobutanecarboxylicacid orcyclobutanecarbonylchloride | 1.60^{ξ} | 400 |
| E16 | 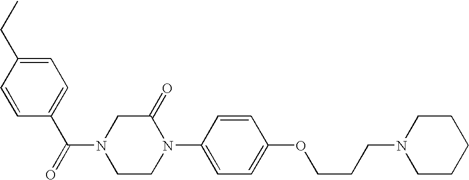 | D22 | 4-ethylbenzoic acid | | |
| E17 | 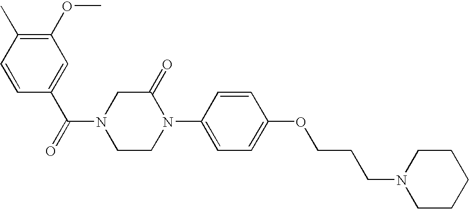 | D22 | 4-methyl-3-(methyloxy)benzoic acid | | |
| E18 | 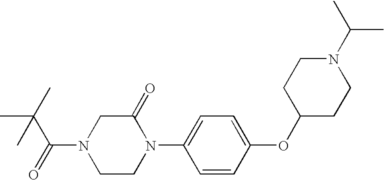 | D7 | pivalic acid | 0.68^{ξ} | 402 |
| E19 | 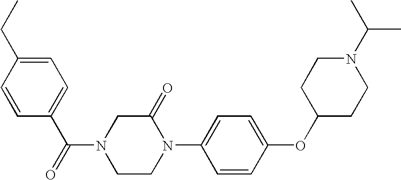 | D7 | 4-ethylbenzoic acid | 0.79^{ξ} | 450 |
| E20 | 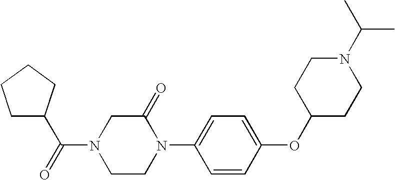 | D7 | cyclopentanecarboxylicacid | 0.68^{ξ} | 414 |
| E21 | 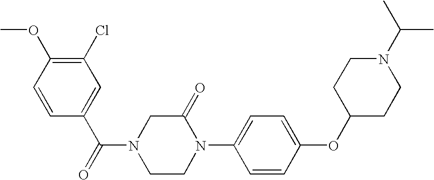 | D7 | 3-chloro-4-(methyloxy)benzoic acid | 0.74^{ξ} | 486,488 |
| E22 | 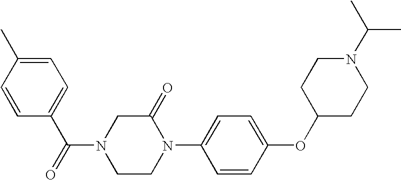 | D7 | 4-methylbenzoic acid | 0.72^{ξ} | 436 |
| E23 | 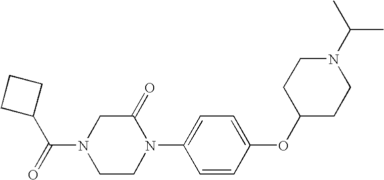 | D7 | cyclobutanecarboxylicacid | 0.64^{ξ} | 400 |
| E24 | 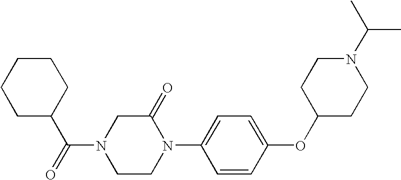 | D7 | cyclohexanecarboxylicacid | 0.74^{ξ} | 428 |
| E25 | 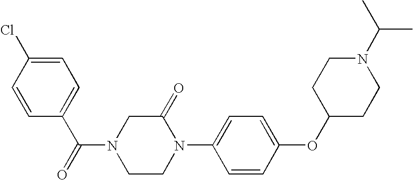 | D7 | 4-chlorobenzoic acid | 0.76^{ξ} | 456,458 |
| E26 | 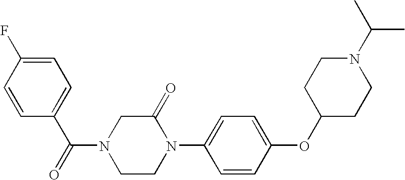 | D7 | 4-fluorobenzoic acid | 0.69^{ξ} | 440 |
| E27 | 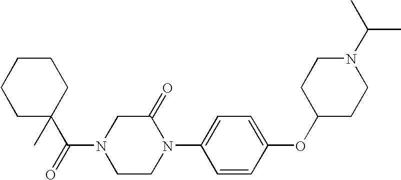 | D7 | 1-methylcyclohexanecarboxylic acid or 1-methylcyclohexanecarbonyl chloride | 1.94* | 442 |
| E28 | 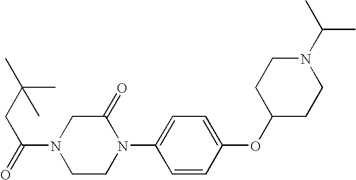 | D7 | 3,3-dimethylbutanoicacid or 3,3-dimethylbutanoyl chloride | 1.73* | 416 |
| E29 | 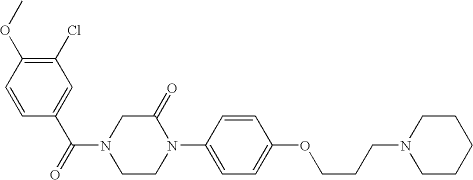 | D22 | 3-chloro-4-(methyloxy)benzoic acid | 1.81* | 486,488 |
| E30 | 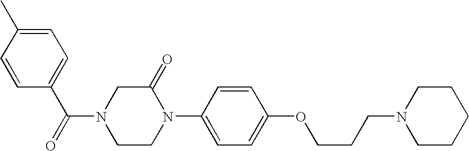 | D22 | 4-methylbenzoic acid | 1.76* | 436 |
| E31 | 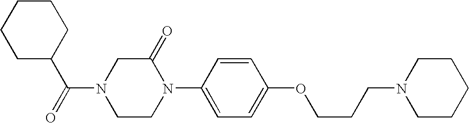 | D22 | cyclohexanecarboxylicacid | 1.81* | 428 |
| E32 | 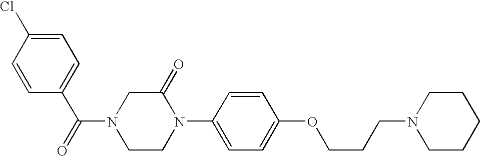 | D22 | 4-chlorobenzoic acid | 1.83* | 456,458 |
| E33 | 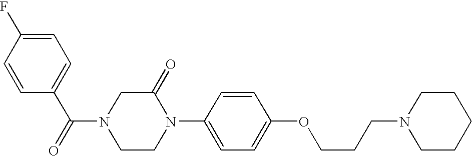 | D22 | 4-fluorobenzoic acid | 2.17 | 440 |
| E34 | 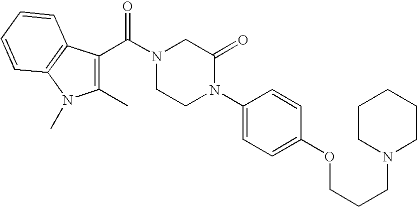 | D22 | 1,2-dimethyl-1H-indole-3-carboxylic acid | 2.16 | 489 |
| E35 | 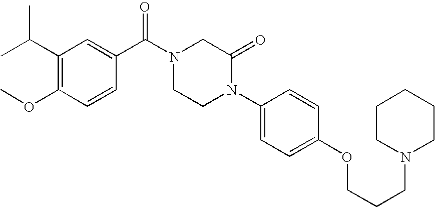 | D22 | 3-(1-methylethyl)-4-(methyloxy)benzoic acid | 2.48 | 494 |
| E36 | 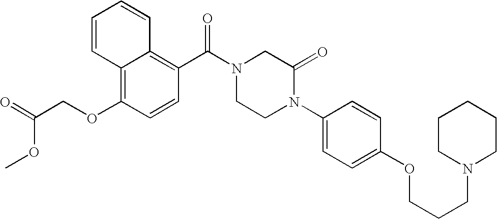 | D22 | 4-{[2-(methyloxy)-2-oxoethyl]oxy}-1-naphthalenecarboxylicacid | 2.36 | 560 |
| E37 | 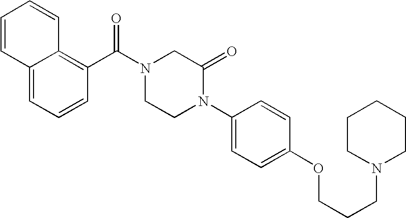 | D22 | 1-naphthalene carboxylicacid | 2.12 | 472 |
| E38 | 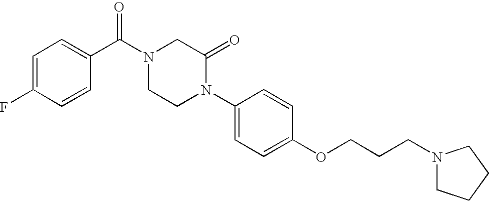 | D16 | 4-fluorobenzoic acid | 2.17 | 426 |
| E39 | 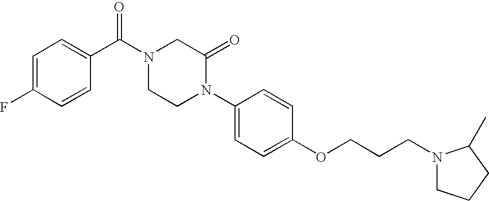 | D18 | 4-fluorobenzoic acid | 2.19 | 440 |
| E40 | 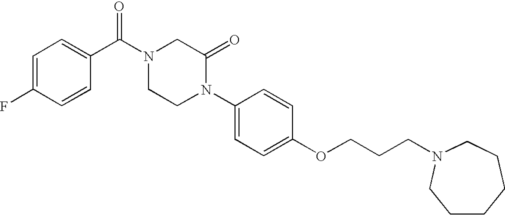 | D24 | 4-fluorobenzoic acid | 2.26 | 454 |
| E41 | 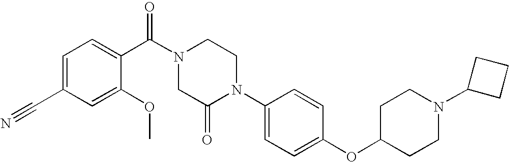 | D5 | 4-cyano-2-(methyloxy)benzoic acid | 2.16 | 489 |
| E42 | 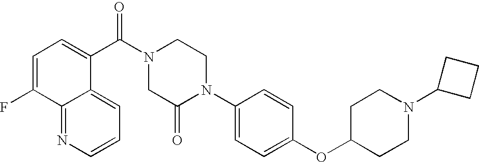 | D5 | 8-fluoro-5-quinolinecarboxylic acid | 2.09 | 503 |
| E43 | 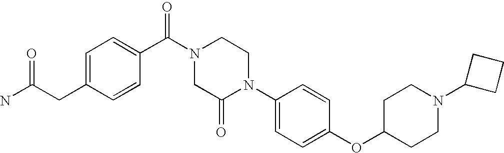 | D5 | 4-(2-amino-2-oxoethyl)benzoic acid | 1.94 | 491 |
| E44 | 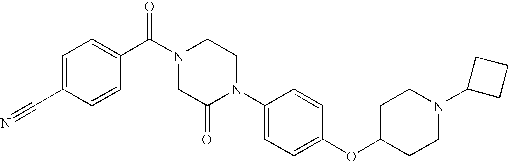 | D5 | 4-cyanobenzoic acid | 2.13 | 459 |
| E45 | 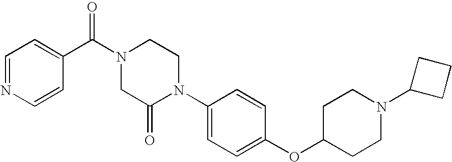 | D5 | 4-pyridinecarboxylic acid | 1.88 | 435 |
| E46 | 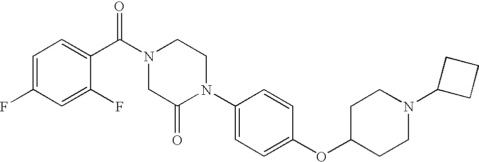 | D5 | 2,4-difluorobenzoic acid | 2.21 | 470 |
| E47 | 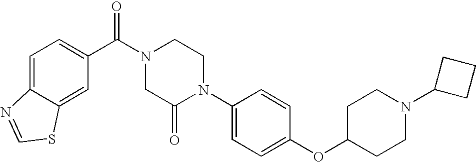 | D5 | 1,3-benzothiazole-6-carboxylic acid | 1.94 | 491 |
| E48 | 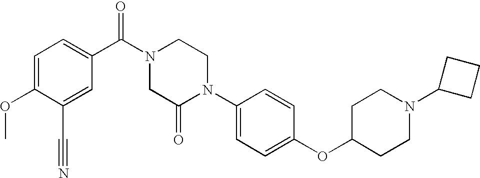 | D5 | 3-cyano-4-(methyloxy)benzoic acid | 2.16 | 489 |
| E49 | 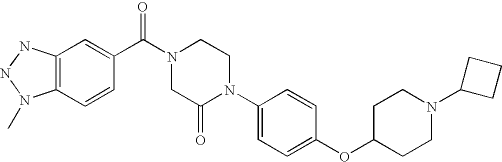 | D5 | 1-methyl-1H-1,2,3-benzotriazole-5-carboxylic acid | 2.01 | 489 |
| E50 | 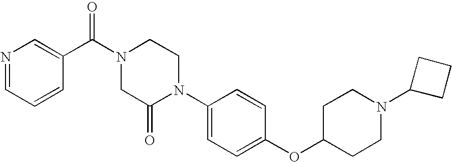 | D5 | 3-pyridinecarboxylic acid | 1.90 | 435 |
| E51 | 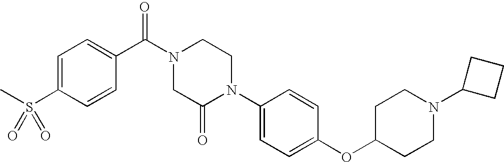 | D5 | 4-(methylsulfonyl)benzoic acid | 2.02 | 512 |
| E52 | 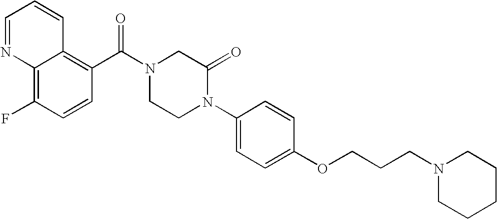 | D22 | 8-fluoro-5-quinolinecarboxylic acid | 2.06 | 491 |
| E53 | 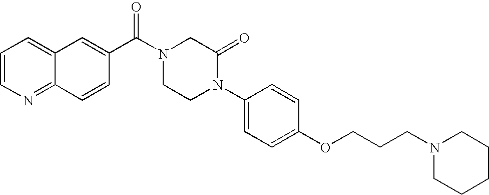 | D22 | 6-quinolinecarboxylicacid | 2.01 | 473 |
| E54 | 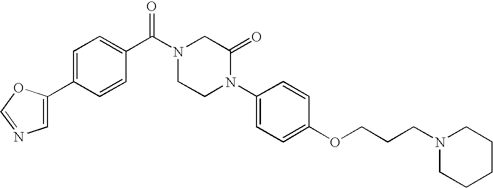 | D22 | 4-(1,3-oxazol-5-yl)benzoic acid | 2.16 | 489 |
| E55 | 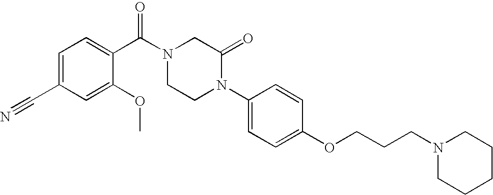 | D22 | 4-cyano-2-(methyloxy)benzoic acid | 2.14 | 477 |
| E56 | 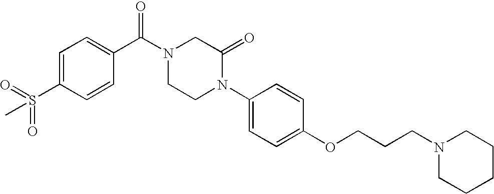 | D22 | 4-(methylsulfonyl)benzoic acid | 2.00 | 500 |
| E57 | 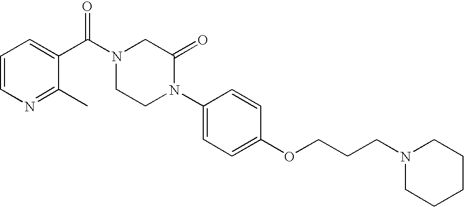 | D22 | 2-methyl-3-pyridinecarboxylic acid | 1.84 | 437 |
| E58 | 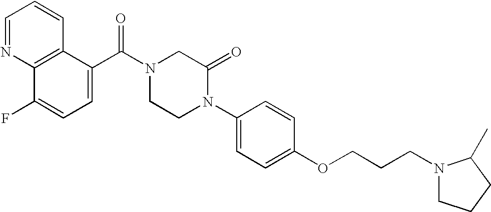 | D22 | 8-fluoro-5-quinolinecarboxylic acid | 2.03 | 491 |
| E59 | 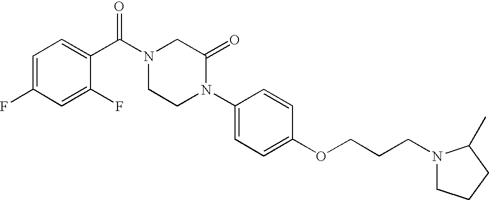 | D18 | 2,4-difluorobenzoic acid | 2.02 | 458 |
| E60 | 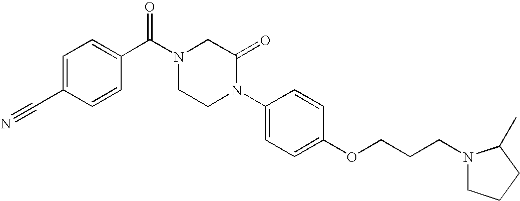 | D18 | 4-cyanobenzoic acid | 1.95 | 447 |
| E61 | 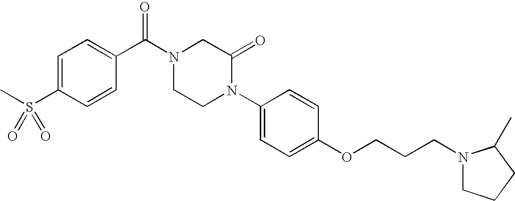 | D18 | 4-(methylsulfonyl)benzoic acid | 1.85 | 500 |
| E62 | 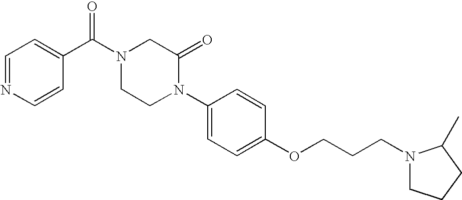 | D18 | 4-pyridinecarboxylic acid | 1.83 | 423 |
| E63 | 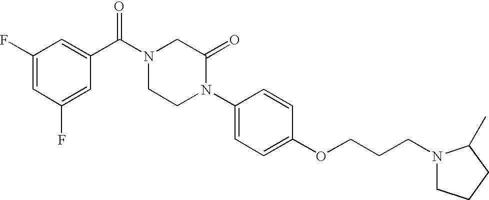 | D18 | 3,5-difluorobenzoic acid | 2.30 | 458 |
| E64 | 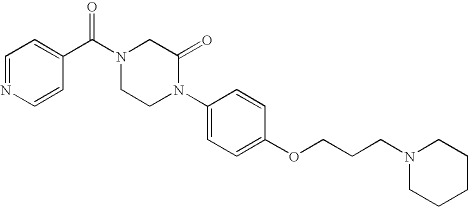 | D22 | 4-pyridinecarboxylic acid | 1.85 | 423 |
| E65 | 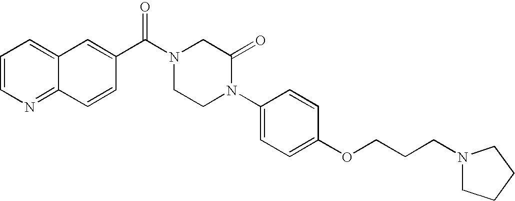 | D16 | 6-quinolinecarboxylicacid | 1.99 | 459 |
| E66 | 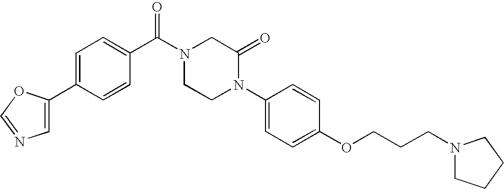 | D16 | 4-(1,3-oxazol-5-yl)benzoic acid | 2.12 | 475 |
| E67 | 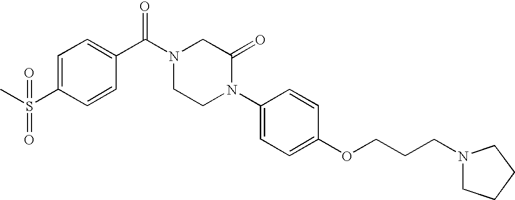 | D16 | 4-(methylsulfonyl)benzoic acid | 1.94 | 486 |
| E68 | 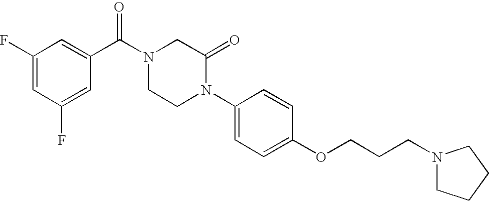 | D16 | 3,5-difluorobenzoic acid | 2.11 | 444 |
| E69 | 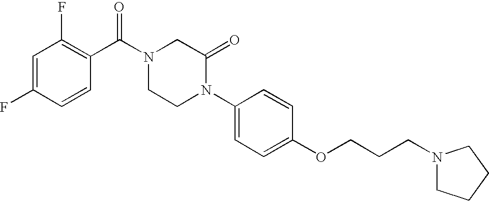 | D16 | 2,4-difluorobenzoic acid | 2.11 | 444 |
| E70 | 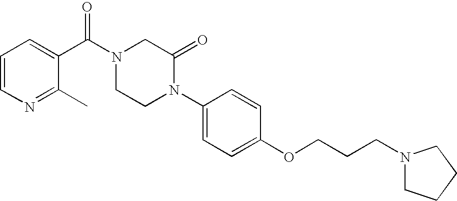 | D16 | 2-methyl-3-pyridinecarboxylic acid | 1.79 | 423 |
| E71 | 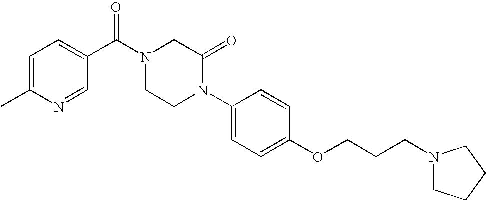 | D16 | 6-methyl-3-pyridinecarboxylic acid | 1.85 | 423 |
| E72 | 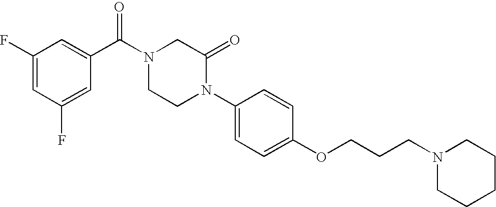 | D22 | 3,5-difluorobenzoic acid | 2.21 | 458 |
| E73 | 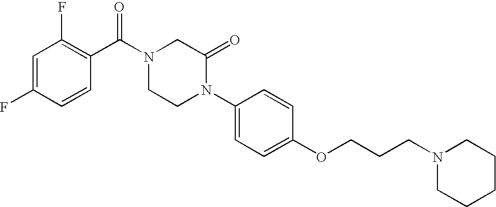 | D22 | 2,4-difluorobenzoic acid | 2.17 | 458 |
| E74 | 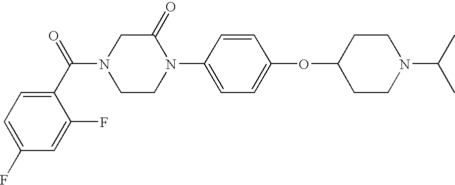 | D7 | 2,4-difluorobenzoic acid | 2.16 | 458 |
| E75 | 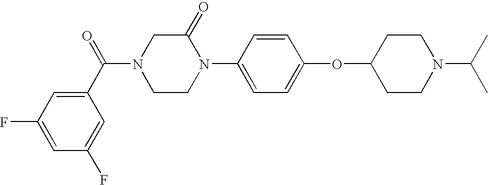 | D7 | 3,5-difiuorobenzoic acid | 2.19 | 458 |
| E76 | 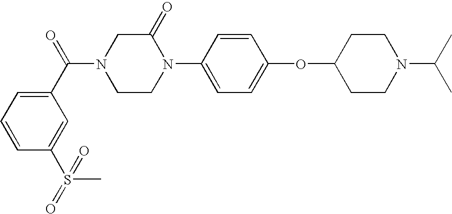 | D7 | 3-(methylsulfonyl)benzoic acid | 1.90 | 500 |
| E77 | 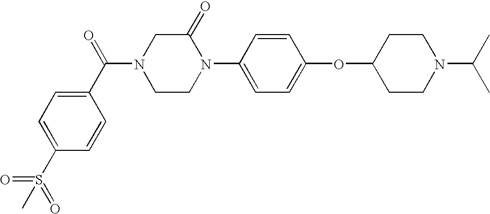 | D7 | 4-(methylsulfonyl)benzoic acid | 1.98 | 500 |
| E78 | 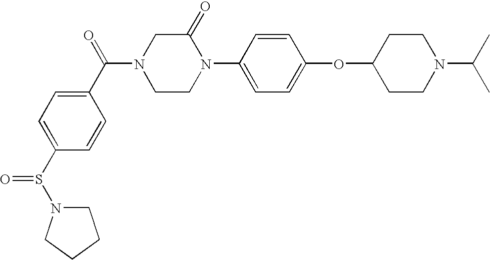 | D7 | 4-(1-pyrrolidinylcarbonyl)benzoic acid | 1.97 | 519 |
| E79 | 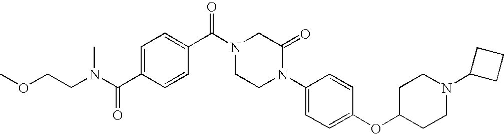 | D5 | 4-({methyl[2-(methyloxy)ethyl]amino}carbonyl)benzoic acid | 2.07 | 549 |
| E80 | 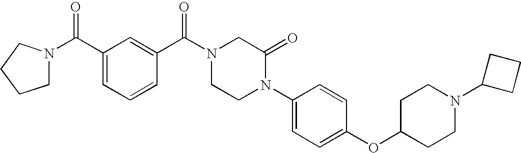 | D5 | 3-(1-pyrrolidinylcarbonyl)benzoic acid | 2.13 | 531 |
| E81 | 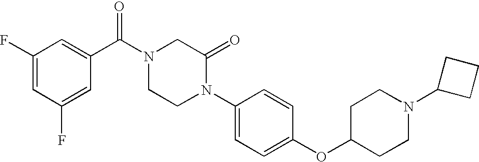 | D5 | 3,5-difluorobenzoic acid | 2.24 | 470 |
| E82 | 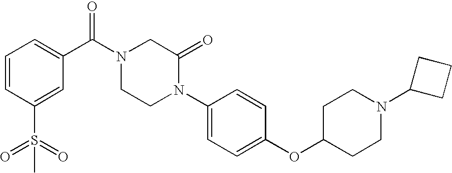 | D5 | 3-(methylsulfonyl)benzoic acid | 2.04 | 512 |
| E83 | 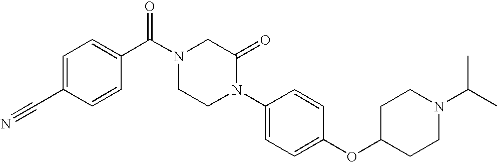 | D7 | 4-cyanobenzoic acid | 2.1 | 447 |
| E84 | 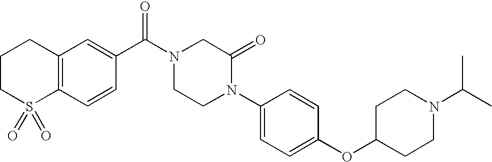 | D7 | 3,4-dihydro-2H-1-benzothiopyran-6-carboxylic acid 1,1-dioxide | 2.0 | 526 |
| E85 | 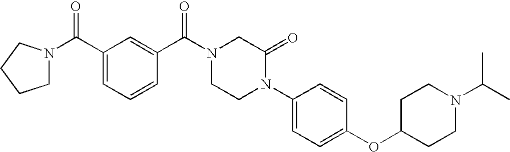 | D7 | 3-(1-pyrrolidinylcarbonyl)benzoic acid | 2.1 | 519 |
| E86 | 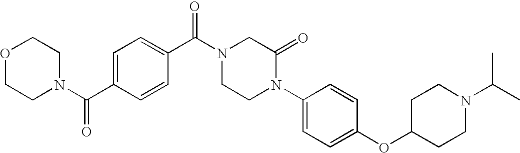 | D7 | 4-(4-morpholinylcarbonyl)benzoic acid | 2.0 | 535 |
| E87 | 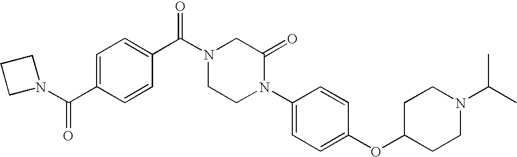 | D7 | 4-(1-azetidinylcarbonyl)benzoic acid | 2.0 | 505 |
| E88 | 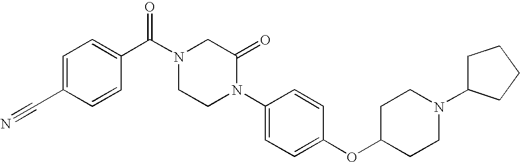 | D9 | 4-cyanobenzoic acid | 2.2 | 473 |
| E89 | 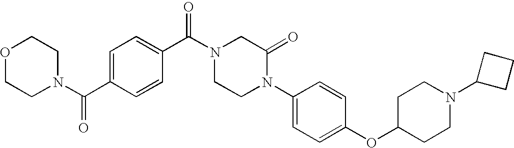 | D5 | 4-(4-morpholinylcarbonyl)benzoic acid | 1.99 | 547 |
| E90 | 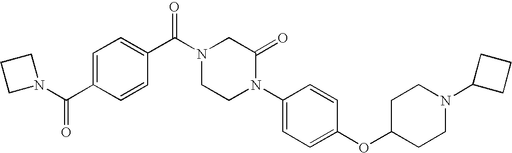 | D5 | 4-(1-azetidinylcarbonyl)benzoic acid | 2.03 | 517 |
| E91 | 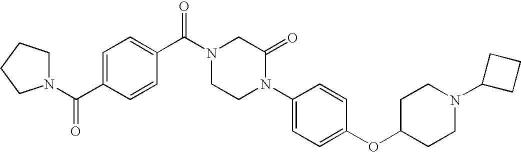 | D5 | 4-(1-pyrrolidinylcarbonyl)benzoic acid | 2.12 | 531 |
| E92 | 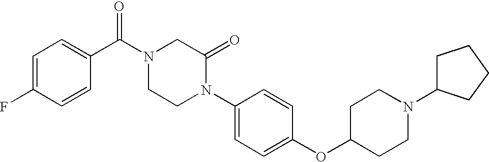 | D9 | 4-fluorobenzoic acid | 2.28 | 466 |
| E93 | 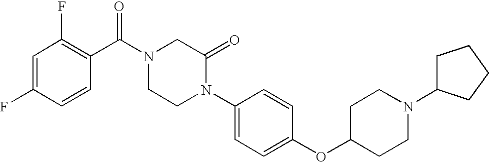 | D9 | 2,4-difluorobenzoic acid | 2.29 | 484 |
| E94 | 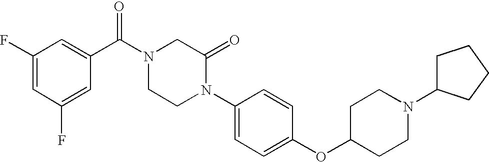 | D9 | 3,5-difluorobenzoic acid | 2.33 | 484 |
| E95 | 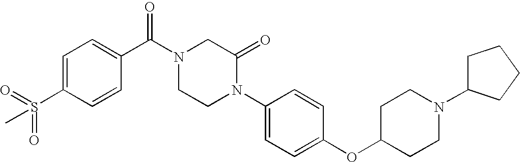 | D9 | 4-(methylsulfonyl)benzoic acid | 2.09 | 526 |
| E96 | 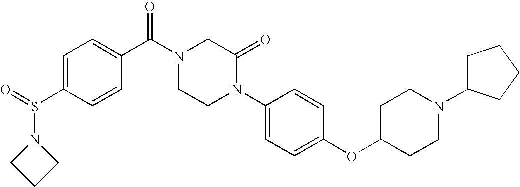 | D9 | 4-(1-azetidinylcarbonyl)benzoic acid | 2.10 | 531 |
| E97 | 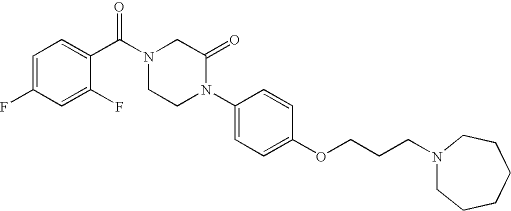 | D24 | 2,4-difluorobenzoic acid | 2.3 | 472 |
| E98 | 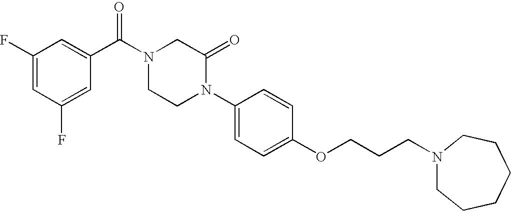 | D24 | 3,5-difluorobenzoic acid | 2.3 | 472 |
| E99 | 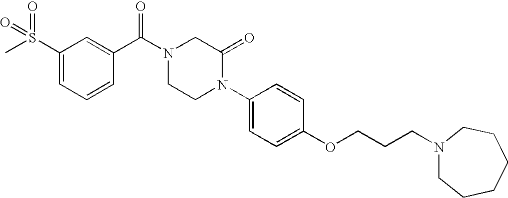 | D24 | 3-(methylsulfonyl)benzoic acid | 2.1 | 514 |
| E100 | 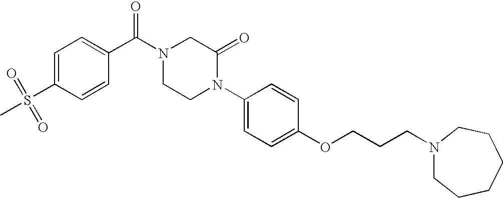 | D24 | 4-(methylsulfonyl)benzoic acid | 2.1 | 514 |
| E101 | 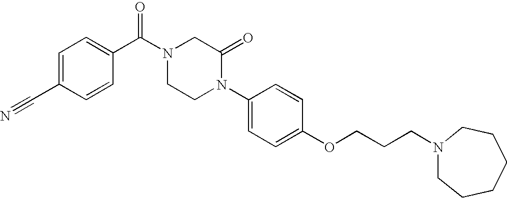 | D24 | 4-cyanobenzoic acid | 2.2 | 461 |
| E102 | 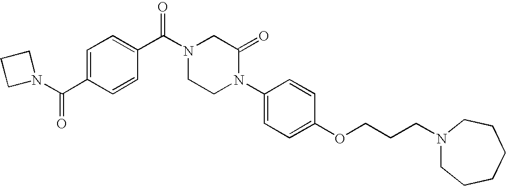 | D24 | 4-(1-azetidinylcarbonyl)benzoic acid | 2.13 | 519 |
| E103 | 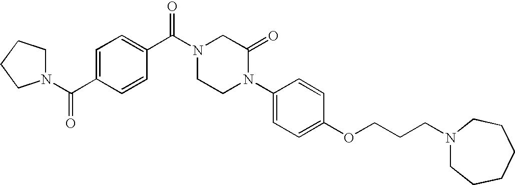 | D24 | 4-(1-pyrrolidinylcarbonyl)benzoic acid | 2.17 | 533 |
| E104 | 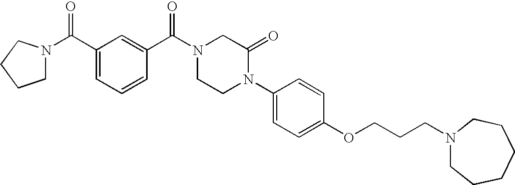 | D24 | 3-(1-pyrrolidinylcarbonyl)benzoic acid | 2.2 | 533 |
| E105 | 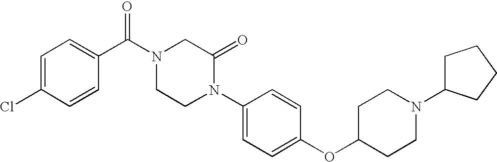 | D9 | 4-chlorobenzoic acid | 2.40 | 482484 |
| E106 | 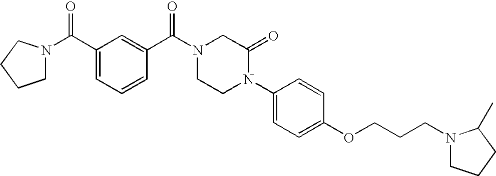 | D18 | 3-(1-pyrrolidinylcarbonyl)benzoic acid | 2.09 | 519 |
| E107 | 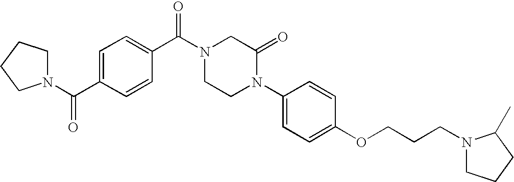 | D18 | 4-(1-pyrrolidinylcarbonyl)benzoic acid | 2.08 | 519 |
| E108 | 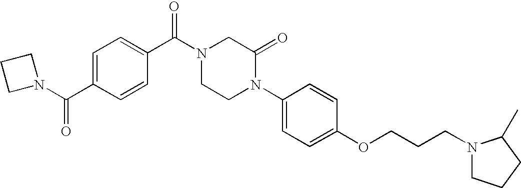 | D18 | 4-(1-azetidinylcarbonyl)benzoic acid | 2.05 | 505 |
| E109 | 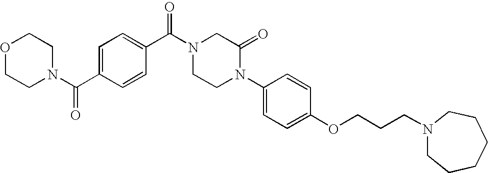 | D24 | 4-(4-morpholinylcarbonyl)benzoic acid | 2.12 | 549 |
| E110 | 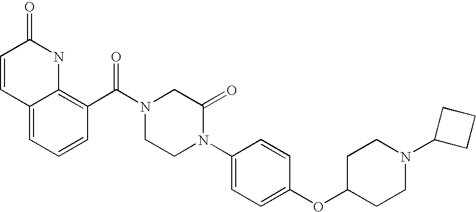 | D5 | 2-oxo-1,2-dihydro-8-quinolinecarboxylic acid | 2.0 | 501 |
| E111 | 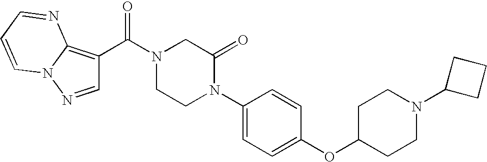 | D5 | pyrazolo[1,5-a]pyrimidine-3-carboxylic acid | 1.9 | 475 |
| E112 | 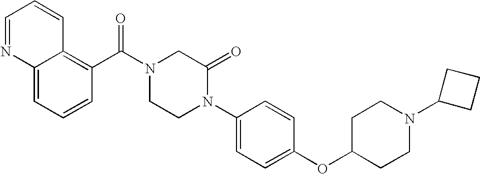 | D5 | 5-quinolinecarboxylicacid | 2.0 | 485 |
| E113 | 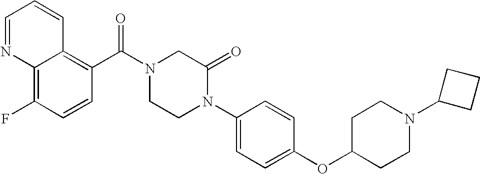 | D5 | 8-fluoro-5-quinolinecarboxylic acid | 2.1 | 503 |
| E114 | 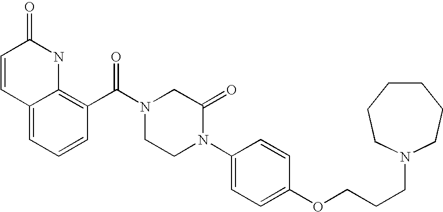 | D24 | 2-oxo-1,2-dihydro-8-quinolinecarboxylic acid | 2.2 | 502 |
| E115 | 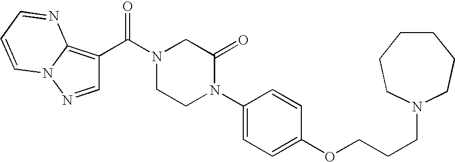 | D24 | pyrazolo[1,5-a]pyrimidine-3-carboxylic acid | 2.0 | 477 |
| E116 | 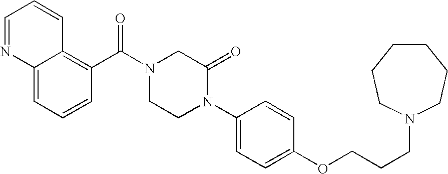 | D24 | 5-quinolinecarboxylicacid | 2.1 | 487 |
| E117 | 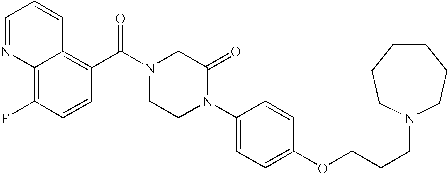 | D24 | 8-fluoro-5-quinolinecarboxylic acid | 2.1 | 505 |
| E118 | 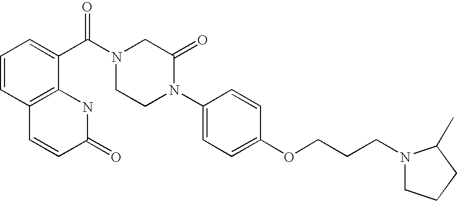 | D18 | 2-oxo-1,2-dihydro-8-quinolinecarboxylic acid | 2.13 | 489 |
| E119 | 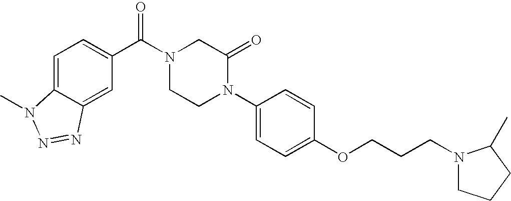 | D18 | 1-methyl-1H-1,2,3-benzotriazole-5-carboxylic acid | 1.98 | 477 |
| E120 | 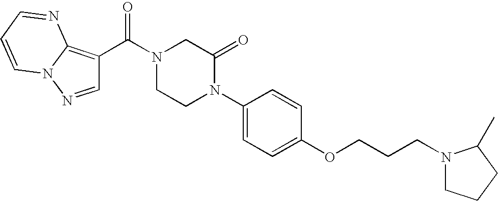 | D18 | pyrazolo[1,5-a]pyrimidine-3-carboxylic acid | 1.86 | 463 |
| E121 | 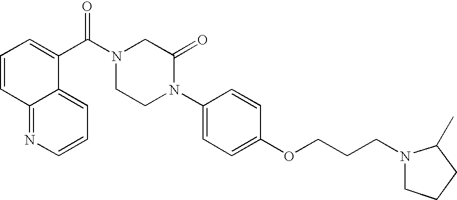 | D18 | 5-quinolinecarboxylicacid | 1.98 | 473 |
| E122 | 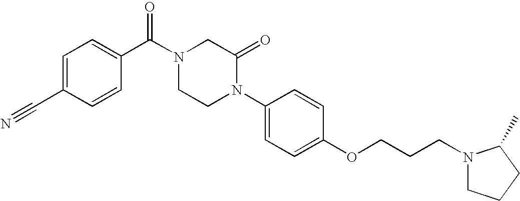 | D26 | 4-cyanobenzoic acid | 2.11 | 447 |
| E123 | 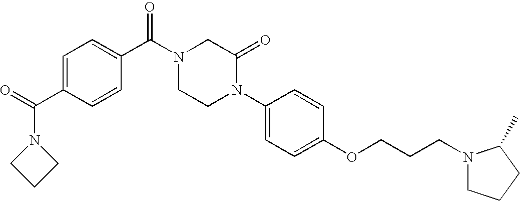 | D26 | 4-(1-azetidinylcarbonyl)benzoic acid | 2.01 | 505 |
| E124 | 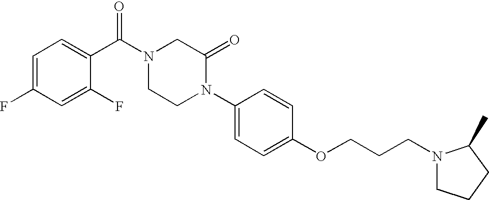 | D20 | 2,4-difluorobenzoic acid | 2.14 | 458 |
| E125 | 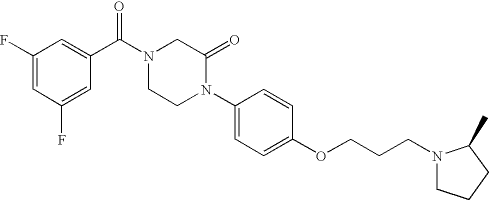 | D20 | 3,5-difluorobenzoic acid | 2.19 | 458 |
| E126 | 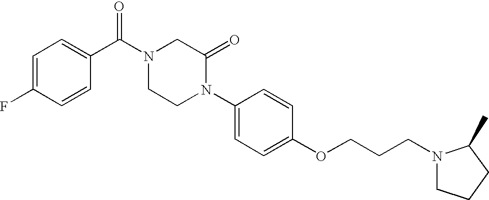 | D20 | 4-fluorobenzoic acid | 2.14 | 440 |
| E127 | 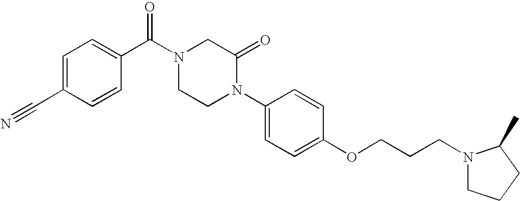 | D20 | 4-cyanobenzoic acid | 2.06 | 447 |
| E128 | 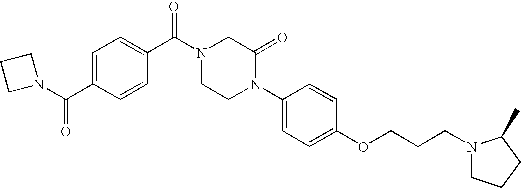 | D20 | 4-(1-azetidinylcarbonyl)benzoic acid | 1.99 | 505 |
| E129 | 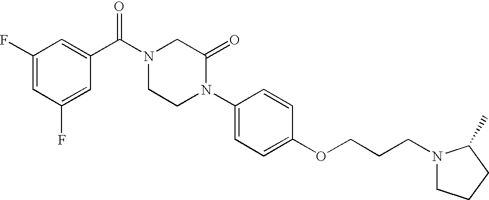 | D26 | 3,5-difluorobenzoic acid | 2.22 | 458 |
| E130 | 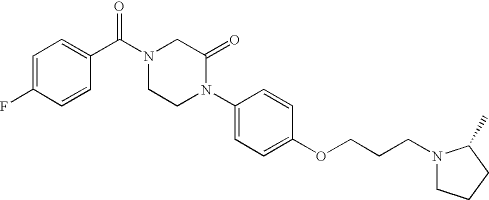 | D26 | 4-fluorobenzoic acid | 2.14 | 440 |
| E131 | 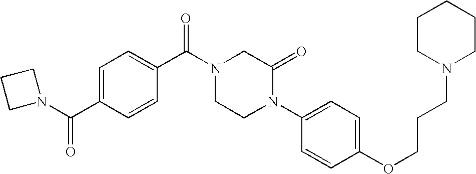 | D22 | 4-(1-azetidinylcarbonyl)benzoic acid | 2.0 | 505 |
| E132 | 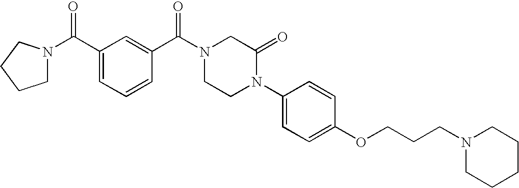 | D22 | 3-(1-pyrrolidinylcarbonyl)benzoic acid | 2.1 | 519 |
| E133 | 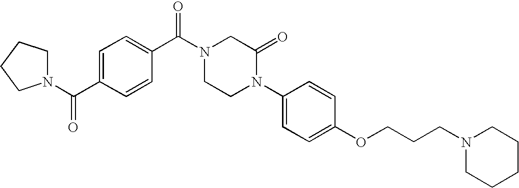 | D22 | 4-(1-pyrrolidinylcarbonyl)benzoic acid | 2.1 | 519 |
| E134 | 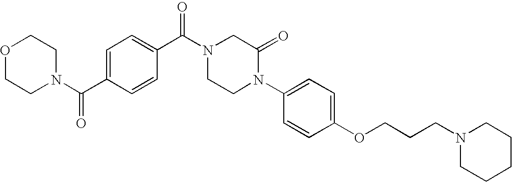 | D22 | 4-(4-morpholinylcarbonyl)benzoic acid | 2.0 | 535 |
| E135 | 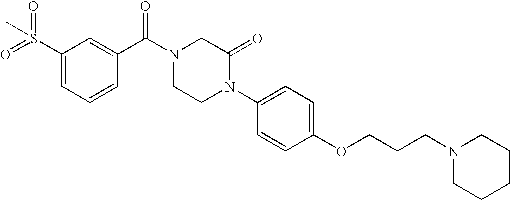 | D22 | 3-(methylsulfonyl)benzoic acid | 2.0 | 500 |
| E136 | 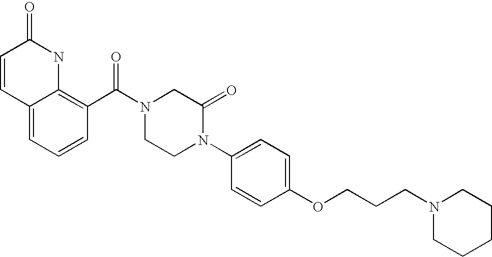 | D22 | 2-oxo-1,2-dihydro-8-quinolinecarboxylic acid | 2.1 | 489 |
| E137 | 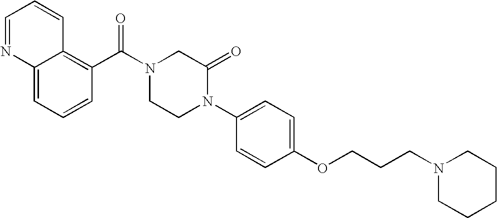 | D22 | 5-quinolinecarboxylicacid | 2.0 | 473 |
| E138 | 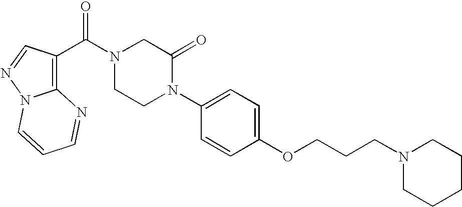 | D22 | pyrazolo[1,5-a]pyrimidine-3-carboxylic acid | 1.9 | 463 |
| E139 | 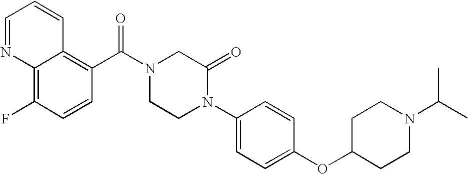 | D7 | 8-fluoro-5-quinolinecarboxylic acid | 2.08 | 491 |
| E140 | 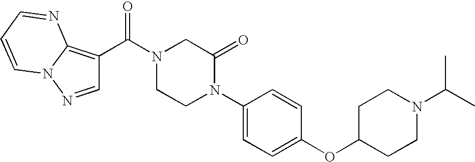 | D7 | pyrazolo[1,5-a]pyrimidine-3-carboxylic acid | 1.93 | 463 |
| E141 | 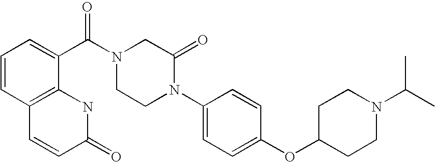 | D7 | 2-oxo-1,2-dihydro-8-quinolinecarboxylic acid | 2.08 | 489 |
| E142 | 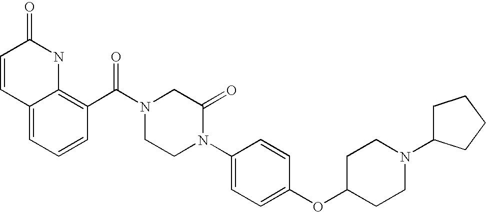 | D9 | 2-oxo-1,2-dihydro-8-quinolinecarboxylic acid | 2.14 | 515 |
| E143 | 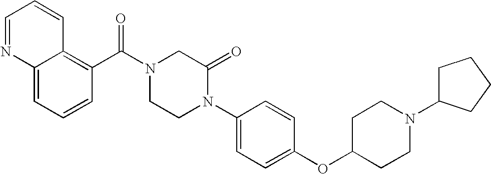 | D9 | 5-quinolinecarboxylicacid | 2.0 | 499 |
| E144 | 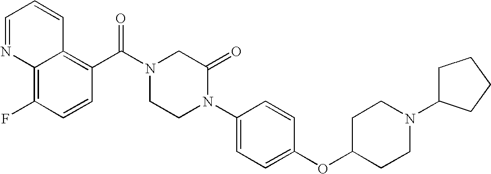 | D9 | 8-fluoro-5-quinolinecarboxylic acid | 2.14 | 517 |
| E145 | 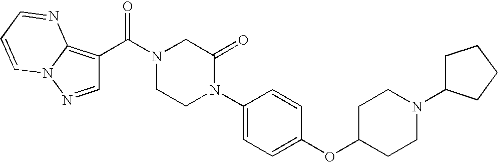 | D9 | pyrazolo[1,5-a]pyrimidine3-carboxylic acid | 2.0 | 489 |
| E146 | 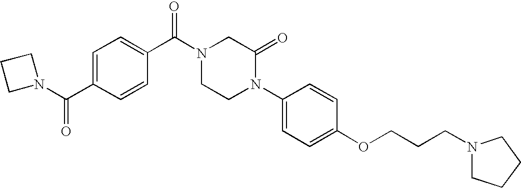 | D16 | 4-(1-azetidinylcarbonyl)benzoic acid | 1.96 | 491 |
| E147 | 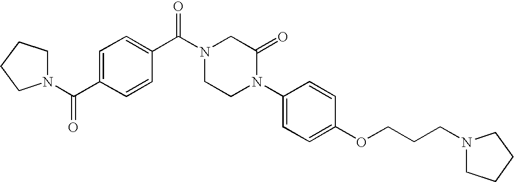 | D16 | 4-(1-pyrrolidinylcarbonyl)benzoic acid | 2.06 | 505 |
| E148 | 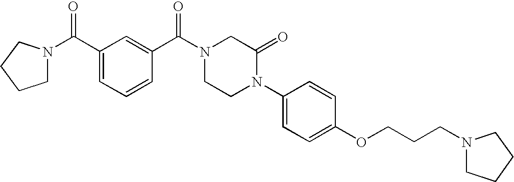 | D16 | 3-(1-pyrrolidinylcarbonyl)benzoic acid | 2.02 | 505 |
| E149 | 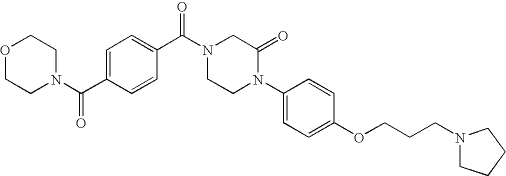 | D16 | 4-(4-morpholinylcarbonyl)benzoic acid | 1.95 | 521 |
| E150 | 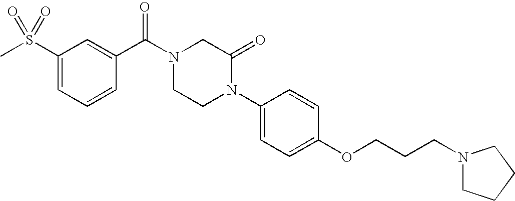 | D16 | 3-(methylsulfonyl)benzoic acid | 1.95 | 486 |
| E151 | 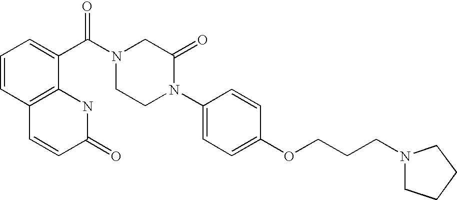 | D16 | 2-oxo-1,2-dihydro-8-quinolinecarboxylic acid | 2.01 | 475 |
| E152 | 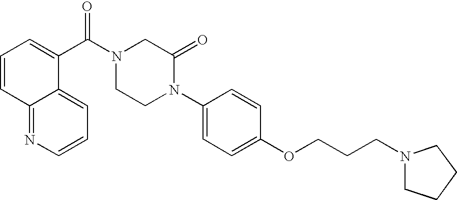 | D16 | 5-quinolinecarboxylicacid | 1.97 | 459 |
| E153 | 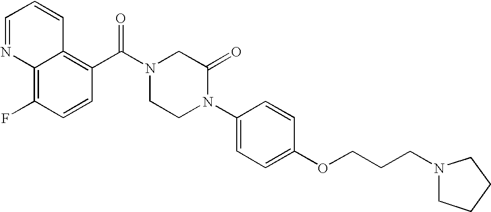 | D16 | 8-fluoro-5-quinolinecarboxylic acid | 2.02 | 477 |
| E154 | 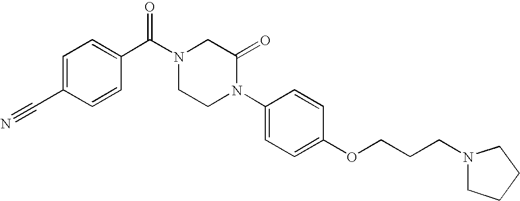 | D16 | 4-cyanobenzoic acid | 2.05 | 433 |
| E155 | 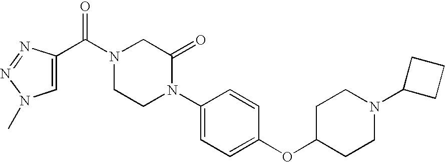 | D5 | 1-methyl-1H-1,2,3-triazole-4-carboxylic acid | 1.91 | 439 |
| E156 | 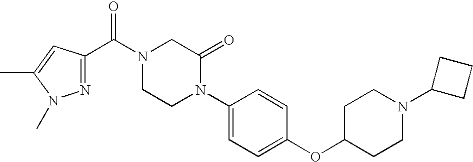 | D5 | 1,5-dimethyl-1H-pyrazole-3-carboxylicacid | 2.00 | 452 |
| E157 | 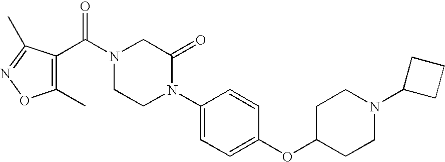 | D5 | 3,5-dimethyl-4-isoxazolecarboxytic acid | 1.94 | 453 |
| E158 | 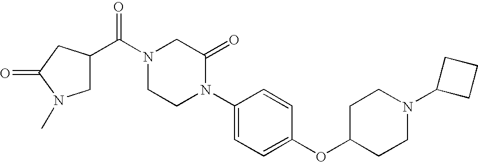 | D5 | 1-methyl-5-oxo-3-pyrrolidinecarboxylic acid | 1.78 | 455 |
| E159 | 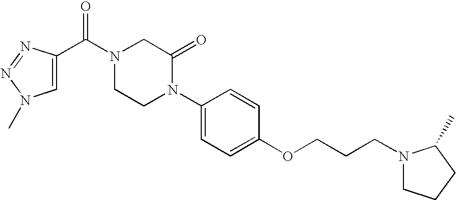 | D26 | 1-methyl-1H-1,2,3-triazole-4-carboxylic acid | 1.85 | 427 |
| E160 | 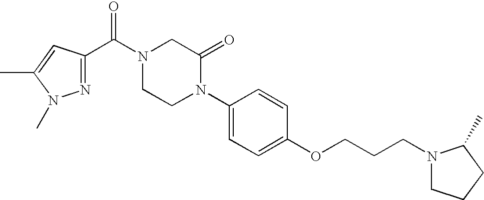 | D26 | 1,5-dimethyl-1H-pyrazole-3-carboxylicacid | 1.99 | 440 |
| E161 | 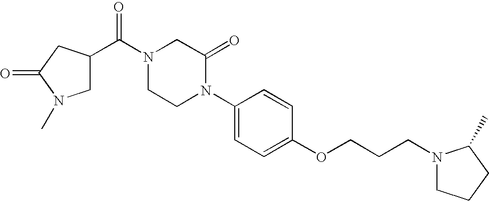 | D26 | 1-methyl-5-oxo-3-pyrrolidinecarboxylic acid | 1.78 | 443 |
| E162 | 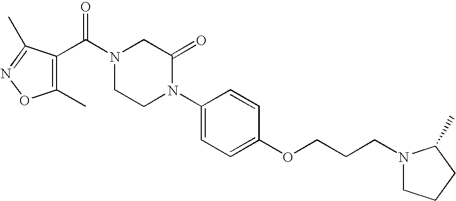 | D26 | 3,5-dimethyl-4-isoxazolecarboxylic acid | 1.94 | 441 |
| E163 | 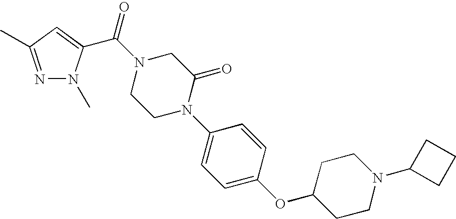 | D5 | 1,3-dimethyl-1H-pyrazole-5-carboxylic acid | 2.03 | 452 |
| E164 | 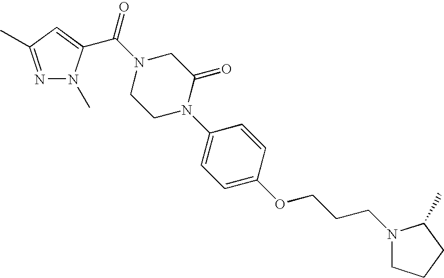 | D26 | 1,3-dimethyl-1H-pyrazole-5-carboxylicacid | 2.00 | 440 |
| E165 | 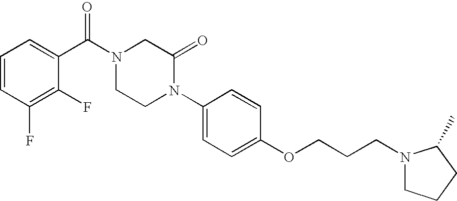 | D26 | 2,3-difluorobenzoic acid | 2.18 | 458 |
| E166 | 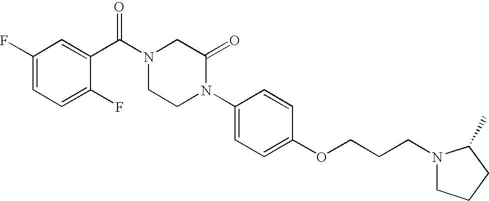 | D26 | 2,5-difluorobenzoic acid | 2.12 | 458 |
| E167 | 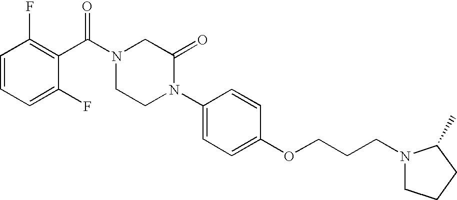 | D26 | 2,6-difluorobenzoic acid | 2.12 | 458 |
| E168 | 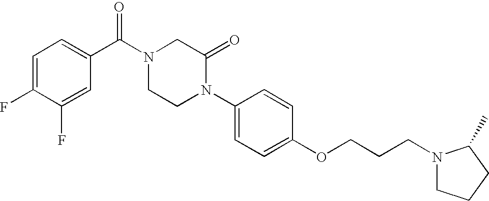 | D26 | 3,4-difluorobenzoic acid | 2.2 | 458 |
| E169 | 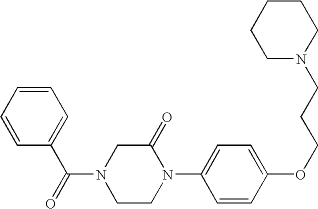 | D22 | benzoic acid | 2.07 | 422 |
| E170 | 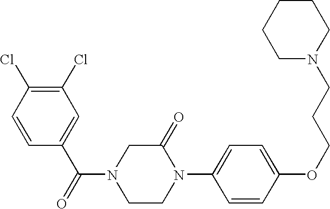 | D22 | 3,4-dichlorobenzoic acid | | |
| E171 | 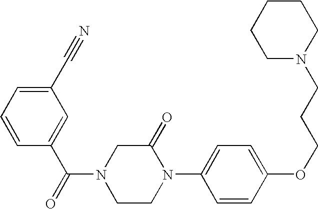 | D22 | 3-cyanobenzoic acid | | |
| E172 | 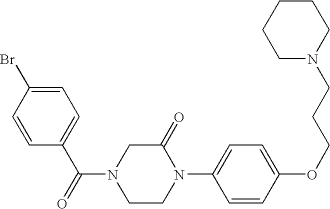 | D22 | 4-bromobenzoic acid | | |
| E173 | 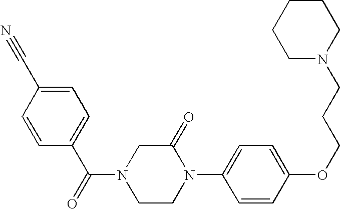 | | 4-cyanobenzoic acid | | |
| E174 | 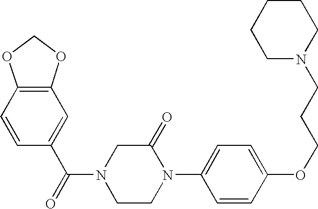 | D22 | 1,3-benzodioxole-5-carboxylic acid | 1.52* | 466 |
| E175 | 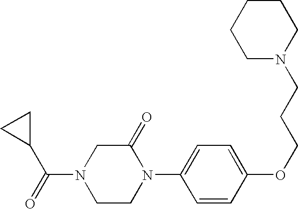 | D22 | cyclopropanecarboxylicacid orcycopropanecaronylchloride | | |

Other compounds of formula (I) that have been prepared by the above general acylation procedure include E176 to E180:

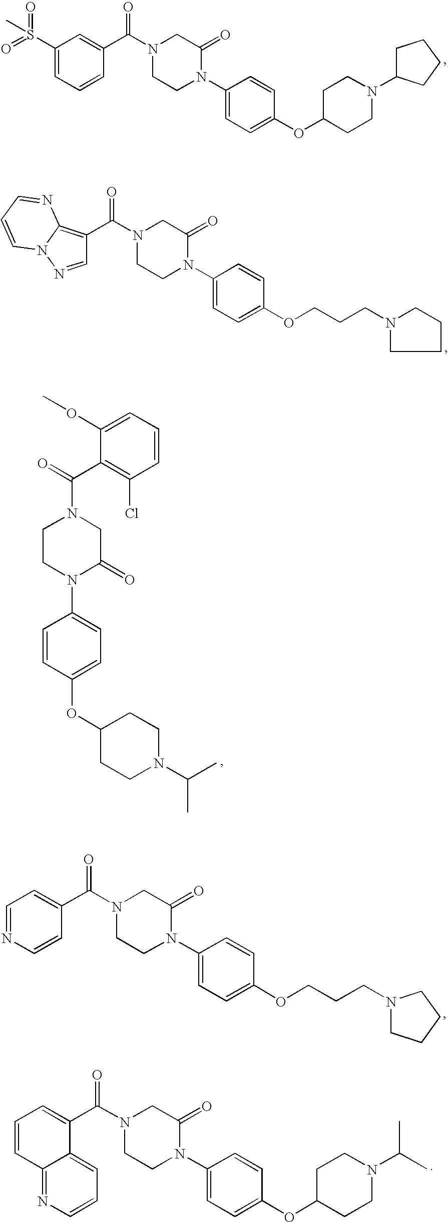

### Biological Data

Compounds of the invention may be tested for in vitro biological activity in accordance with the following or similar assays:

### H1 Receptor Cell Line Generation and FLIPR Assay Protocol

### 1. Generation of Histamine H1 Cell Line

The human H1 receptor was cloned using known procedures described in the literature [Biochem. Biophys. Res. Commun., 201(2):894, (1994)]. Chinese hamster ovary cells stably expressing the human H1 receptor were generated according to known procedures described in the literature [Br. J. Pharmacol., 117(6):1071, (1996)].

### Histamine H1 Functional Antagonist Assay

The histamine H1 cell line was seeded into non-coated black-walled clear bottom 384-well tissue culture plates in alpha minimum essential medium (Gibco/Invitrogen, cat no. 22561-021), supplemented with 10% dialysed foetal calf serum (Gibco/Invitrogen cat no. 12480-021) and 2 mM L-glutamine (Gibco/Invitrogen cat no 25030-024) and maintained overnight at 5% CO₂, 37° C.

Excess medium was removed from each well to leave 10 μl. 30 μl loading dye (250 μM Brilliant Black, 2 μM Fluo-4 diluted in Tyrodes buffer+probenecid (145 mM NaCl, 2.5 mM KCl, 10 mM HEPES, 10 mM D-glucose, 1.2 mM MgCl₂, 1.5 mM CaCl₂, 2.5 mM probenecid, pH adjusted to 7.40 with NaOH 1.0 M)) was added to each well and the plates were incubated for 60 min at 5% CO₂, 37° C.

10 μl of test compound, diluted to the required concentration in Tyrodes buffer+probenecid (or 10 μl Tyrodes buffer+probenecid as a control) was added to each well and the plate incubated for 30 min at 37° C., 5% CO₂. The plates were then placed into a FLIPR™ (Molecular Devices, UK) to monitor cell fluorescence (λₑₓ=488 nm, λ_{EM}=540 nm) in the manner described in Sullivan et al., (In: Lambert DG (ed.), Calcium Signaling Protocols, New Jersey: Humana Press, 1999, pp. 125-136) before and after the addition of 10 μl histamine at a concentration that results in the final assay concentration of histamine being EC₈₀.

Functional antagonism is indicated by a suppression of histamine-induced increase in fluorescence, as measured by the FLIPR™ system (Molecular Devices). By means of concentration effect curves, functional affinities are determined using standard pharmacological mathematical analysis.

### 2. H3 Receptor Cell Line Generation, Membrane Preparation and Functional GTPγS Assay Protocols

### Generation of Histamine H3 Cell Line

The histamine H3 cDNA was isolated from its holding vector, pcDNA3.1 TOPO (InVitrogen), by restriction digestion of plasmid DNA with the enzymes BamH1 and Not-1 and ligated into the inducible expression vector pGene (InVitrogen) digested with the same enzymes. The GeneSwitch™ system (a system where in transgene expression is switched off in the absence of an inducer and switched on in the presence of an inducer) was performed as described in U.S. Pat. Nos. 5,364,791; 5,874,534; and 5,935,934. Ligated DNA was transformed into competent DH5α E. coli host bacterial cells and plated onto Luria Broth (LB) agar containing Zeocin™ (an antibiotic which allows the selection of cells expressing the sh ble gene which is present on pGene and pSwitch) at 50 μgml⁻¹. Colonies containing the re-ligated plasmid were identified by restriction analysis. DNA for transfection into mammalian cells was prepared from 250 ml cultures of the host bacterium containing the pGeneH3 plasmid and isolated using a DNA preparation kit (Qiagen Midi-Prep) as per manufacturers guidelines (Qiagen).

CHO K1 cells previously transfected with the pSwitch regulatory plasmid (InVitrogen) were seeded at 2×10⁶ cells per T75 flask in Complete Medium, containing Hams F12 (GIBCOBRL, Life Technologies) medium supplemented with 10% v/v dialysed foetal bovine serum, L-glutamine, and hygromycin (100 μgml⁻¹), 24 h prior to use. Plasmid DNA was transfected into the cells using Lipofectamine plus according to the manufacturers guidelines (InVitrogen). 48 h post transfection cells were placed into complete medium supplemented with 500 μgml⁻¹ Zeocin™.

10-14 days post selection 10 nM Mifepristone (InVitrogen), was added to the culture medium to induce the expression of the receptor. 18 h post induction cells were detached from the flask using ethylenediamine tetra-acetic acid (EDTA; 1:5000; InVitrogen), following several washes with phosphate buffered saline pH 7.4 and resuspended in Sorting Medium containing Minimum Essential Medium (MEM), without phenol red, and supplemented with Earles salts and 3% Foetal Clone II (Hyclone). Approximately 1×10⁷ cells were examined for receptor expression by staining with a rabbit polyclonal antibody, 4a, raised against the N-terminal domain of the histamine H3 receptor, incubated on ice for 60 min, followed by two washes in sorting medium. Receptor bound antibody was detected by incubation of the cells for 60 min on ice with a goat anti rabbit antibody, conjugated with Alexa 488 fluorescence marker (Molecular Probes). Following two further washes with Sorting Medium, cells were filtered through a 50 μm Filcon™ (BD Biosciences) and then analysed on a FACS Vantage SE Flow Cytometer fitted with an Automatic Cell Deposition Unit. Control cells were non-induced cells treated in a similar manner. Positively stained cells were sorted as single cells into 96-well plates, containing Complete Medium containing 500 μgml⁻¹ Zeocin™ and allowed to expand before reanalysis for receptor expression via antibody and ligand binding studies. One clone, 3H3, was selected for membrane preparation.

Membrane Preparation from Cultured Cells

All steps of the protocol are carried out at 4° C. and with pre-cooled reagents. The cell pellet is resuspended in 10 volumes of homogenisation buffer (50 mM N-2-hydroxyethylpiperazine-N′-2-ethanesulfonic acid (HEPES), 1 mM ethylenediamine tetra-acetic acid (EDTA), pH 7.4 with KOH, supplemented with 10⁻⁶ M leupeptin (acetyl-leucyl-leucyl-arginal; Sigma L2884), 25 μgml⁻¹ bacitracin (Sigma B0125), 1 mM phenylmethylsulfonyl fluoride (PMSF) and 2×10⁻⁶ M pepstain A (Sigma)). The cells are then homogenised by 2×15 second bursts in a 1 litre glass Waring blender, followed by centrifugation at 500 g for 20 min. The supernatant is then spun at 48,000 g for 30 min. The pellet is resuspended in homogenisation buffer (4× the volume of the original cell pellet) by vortexing for 5 seconds, followed by homogenisation in a Dounce homogeniser (10-15 strokes). At this point the preparation is aliquoted into polypropylene tubes and stored at −80° C.

### Histamine H3 Functional Antagonist Assay

For each compound being assayed, in a solid white 384 well plate, is added:—

(a) 0.5 μl of test compound diluted to the required concentration in DMSO (or 0.5 μl DMSO as a control);
(b) 30 μl bead/membrane/GDP mix prepared by mixing Wheat Germ Agglutinin Polystyrene LeadSeeker® (WGA PS LS) scintillation proximity assay (SPA) beads with membrane (prepared in accordance with the methodology described above) and diluting in assay buffer (20 mM N-2-Hydroxyethylpiperazine-N′-2-ethanesulfonic acid (HEPES)+100 mM NaCl+10 mM MgCl₂, pH7.4 NaOH) to give a final volume of 30 μl which contains 5 μg protein and 0.25 mg bead per well, incubating at room temperature for 60 min on a roller and, just prior to addition to the plate, adding 10 μM final concentration of guanosine 5′ diphosphate (GDP) (Sigma; diluted in assay buffer);
(c) 15 μl 0.38 nM [³⁵S]-GTPγS (Amersham; Radioactivity concentration=37 MBqml⁻¹; Specific activity=1160 Cimmol⁻¹), histamine (at a concentration that results in the final assay concentration of histamine being EC₈₀).

After 2-6 h, the plate is centrifuged for 5 min at 1500 rpm and counted on a Viewlux counter using a 613/55 filter for 5 minplate-1. Data is analysed using a 4-parameter logistical equation. Basal activity used as minimum i.e. histamine not added to well.

Bioavailability and CNS penetration of compounds of the invention may be assayed in the following, or similar, assays.

### 1. CNS Penetration Method

Compounds are dosed intravenously at a nominal dose level of 1 mgkg⁻¹ to male CD Sprague Dawley rats. Compounds are formulated in 5% DMSO/45% PEG200/50% water. Blood samples are taken under terminal anaesthesia with isoflurane at 5 min post-dose and the brains are also removed for assessment of brain penetration. Blood samples are taken directly into heparinised tubes. Blood samples are prepared for analysis using protein precipitation, and brain samples are prepared using extraction of drug from brain by homogenisation and subsequent protein precipitation. The concentration of parent drug in blood and brain extracts is determined by quantitative LC-MS/MS analysis using compound-specific mass transitions.

### 2. Rat Pharmacokinetics Method

Compounds are dosed to male CD Sprague Dawley rats by single intravenous or oral administration at a nominal dose level of 1 mgkg⁻¹ and 3 mgkg⁻¹ respectively. Compounds are formulated in 5% DMSO/45% PEG200/50% water. An intravenous profile is obtained by taking serial or terminal blood samples at 0.083, 0.25, 0.5, 1, 2, 4, and 7 h post-dose. An oral profile is obtained by taking serial or terminal blood samples at 0.25, 0.5, 1, 2, 4, 7 and 12 h post-dose. Blood samples are taken directly into heparinised tubes. Blood samples are prepared by protein precipitation and are subjected to quantitative analysis by LC-MS/MS using compound-specific mass transitions. Drug concentration-time profiles are generated and non-compartmental PK analysis is used to generate estimates of half-life, clearance, volume of distribution and oral bioavailability.

### 3. Dog Pharmacokinetics Method

Compounds are dosed to male Beagle dogs by single intravenous or oral administration at a nominal dose level of 1 mgkg⁻¹ and 2 mgkg⁻¹ respectively. The study is carried out according to a crossover design, such that the same dog is used for both dosing events and the dosing events occur one week apart. Compounds are formulated in 5% DMSO/45% Peg200/50% water. An intravenous profile is obtained by taking serial blood samples at 0.083, 0.25, 0.5, 0.75, 1, 2, 4, 6 & 12 h post-dose. An oral profile is obtained by taking serial blood samples at 0.25, 0.5, 0.75, 1, 2, 4, 6, 12 & 24 h post-dose. Blood samples are taken directly into heparinised tubes. Blood samples are prepared by protein precipitation and are subjected to quantitative analysis by LC-MS/MS using compound-specific mass transitions. Drug concentration-time profiles are generated and non-compartmental PK analysis is used to generate estimates of half-life, clearance, volume of distribution and oral bioavailability.

### Results

In the above assays or similar assays Examples E1 to E175 had an average pKᵢ (pK_{b}) at H3 of greater than about 8.0 and at H1 of less than about 6.0.

Examples E176 to E180 had average pKᵢ (pK_{b}) at H3 of greater than about 7.8 and at H1 of less than about 6.0.

The content of all documents including literature references, patents and patent applications referred to hereinabove are to be considered as incorporated in full herein.

What is claimed is:

1. A compound of formula (I) 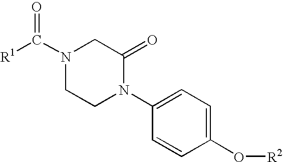
in which
R¹ represents —C₁₋₆alkyl, —C₁₋₆alkoxy, —C₃₋₈cycloalkyl (optionally substituted by C₁₋₆alkyl), heterocyclyl, aryl, heteroaryl, and -aryl-heteroaryl,
wherein independently each of said heterocyclyl, aryl, heteroaryl, and -aryl-heteroaryl of R¹ may be optionally substituted by one or two substituents which may be the same or different selected from C₁₋₆alkyl, (optionally substituted by COOR³ wherein R³ is C₁₋₆alkyl or hydrogen), C₁₋₆alkoxy (optionally substituted by COOR⁴ wherein R⁴ is C₁₋₆alkyl or hydrogen), cyano, oxo, halogen, C₁₋₆alkylsulfonyl, —C₁₋₆alkylCONR⁵R⁶ wherein R⁵ and R⁶ independently represent hydrogen, C₁₋₆alkyl, C₁₋₆alkoxy, C₁₋₆alkylC₁₋₆alkoxy, or together NR⁵R⁶ may form a 4- to 7-membered non-aromatic heterocyclic ring (optionally containing an O or S atom and optionally substituted by C₁₋₆alkyl, halogen or C₁₋₆alkoxy), —CONR⁷R⁸ wherein R⁷ and R⁸ independently represent hydrogen, C₁₋₆alkyl, C₁₋₆alkoxy, C₁₋₆ alkylC₁₋₆alkoxy, or together NR⁷R⁸ may form a 4- to 7-membered non-aromatic heterocyclic ring (optionally containing an 0 or an S atom and optionally substituted by C₁₋₆alkyl, halogen or C₁₋₆alkoxy);
R² represents —(CH₂)ₓ—NR⁹R¹⁰ in which NR⁹R¹⁰ represents an N-linked nitrogen containing heterocyclyl ring (optionally substituted by one or two substituents selected from trifluoromethyl or C₁₋₆alkyl), and x is 2, 3 or 4, or
R² represents the group: 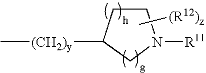
wherein R¹¹ represents C₁₋₆alkyl, C₃₋₈cycloalkyl or C₁₋₆alkyl C₃₋₈cycloalkyl, R¹² represents trifluoromethyl or C₁₋₆ alkyl, z is 0 or 1, y is 0 or 1, and g is 0, 1, or 2 and h is 0, 1, 2, or 3 such that g and h cannot both be 0; or
a salt thereof,
with the proviso that the compound is not 4-[(2,4-difluorophenyl)carbonyl]-1-[4-({3-[2-methyl-1-pyrrolidinyl]propyl}oxy)phenyl]-2-piperazinone, or a salt thereof.

2. A compound according to claim 1 in which R¹ may be optionally substituted by one or two substituents which may be the same or different selected from C₁₋₆alkyl, (optionally substituted by COOR³ wherein R³ is C₁₋₆alkyl or hydrogen), C₁₋₆alkoxy (optionally substituted by COOR⁴ wherein R⁴ is C₁₋₆alkyl or hydrogen), cyano, oxo, C₁₋₆alkylsulfonyl, —C₁₋₆alkylCONR⁵R⁶ wherein R⁵ and R⁶ independently represent hydrogen, C₁₋₆alkyl, C₁₋₆alkoxy, C₁₋₆alkylC₁₋₆alkoxy, or together NR⁵R⁶ may form a 4- to 7-membered non-aromatic heterocyclic ring (optionally containing an O or S atom and optionally substituted by C₁₋₆alkyl, halogen or C₁₋₆alkoxy), —CONR⁷R⁸ wherein R⁷ and R⁸ independently represent hydrogen, C₁₋₆alkyl, C₁₋₆alkoxy, C₁₋₆ alkylC₁₋₆alkoxy, or together NR⁷R³ may form a 4- to 7-membered non-aromatic heterocyclic ring (optionally containing an 0 or an S atom and optionally substituted by C₁₋₆alkyl, halogen or C₁₋₆alkoxy).

3. (canceled)

4. Compound according to claim 1 wherein the salt is a pharmaceutically acceptable salt or solvate.

5. A process for the preparation of a compound of claim 1 which process comprises a process selected from (a) or (b) below and optionally thereafter forming a salt thereof:
(a) reacting a compound of formula (II) 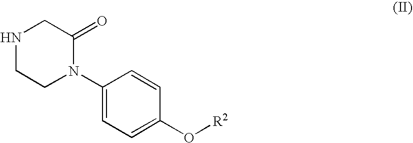
in which R² is as defined for a compound of formula (I), or a salt thereof,
with a compound of formula (III): R¹COOH
in which R¹ is as defined for a compound of formula (I);
(b) interconversion from other compounds of formula (I); or
(c) preparation of a salt of a compound of formula (I).

6-8. (canceled)

9. A composition which comprises a therapeutically effective amount of a compound according to claim 1, or a pharmaceutically acceptable salt thereof, and one or more pharmaceutically acceptable carriers and/or excipients.

10. A composition according to claim 9 which further comprises an H1 receptor antagonist.

11. A combination comprising a compound of claim 1, or a pharmaceutically acceptable salt thereof and a H1 receptor antagonist.

12. The use of a compound according to claim 1 or a pharmaceutically acceptable salt thereof in the manufacture of a medicament for the treatment or prophylaxis of inflammatory and/or allergic disorders.

13. (canceled)

14. A method for the treatment or prophylaxis of inflammatory and/or allergic disorders which comprises administering to a patient in need thereof an effective amount of a compound according to claim 1 or a pharmaceutically acceptable salt thereof.

15. A method according to claim 14 wherein the disorder is allergic rhinitis.
